(19) United States
(12) Patent Application Publication
(10) Pub. No.: US 2026/0011408 A1
(43) Pub. Date: Jan. 8, 2026

(54) Context-Specific Tumor-Only Mutation Classification

(71) Applicants: The Broad Institute, Inc., Cambridge, MA (US); The General Hospital Corporation, Boston, MA (US)

(72) Inventors: Gad A. Getz, Belmont, MA (US); Claudia Lichieh Chu, Cambridge, MA (US); Donald Arthur Stewart, JR., Hingham, MA (US); Andrew James Dunford, East Boston, MA (US); Kristy Lynn Schlueter-Kuck, Needham, MA (US); Amber Marie Pospistle, Huntersville, NC (US)

(73) Assignees: The Broad Institute, Inc., Cambridge, MA (US); The General Hospital Corporation, Boston, MA (US)

(21) Appl. No.: 19/258,562

(22) Filed: Jul. 2, 2025

Related U.S. Application Data
(60) Provisional application No. 63/667,622, filed on Jul. 3, 2024.

(51) Int. Cl. G16B 30/00; G16B 40/20; G16H 70/60

(52) U.S. Cl. CPC G16B 30/00; G16B 40/20; G16H 70/60

(57) ABSTRACT

Context-specific tumor-only mutation classification is described. A mutation classification module may classify a mutation identified in sequencing data from a tumor sample as germline or somatic based on a likelihood ratio relative to a threshold, the likelihood ratio comparing a germline model likelihood of a germline model of the mutation to a somatic model likelihood of a somatic model of the mutation and the threshold calculated based on a context of the mutation. The mutation classification module may output the classification of the mutation.

### RELATED APPLICATION

This application claims priority to U.S. Provisional Patent Application Ser. No. 63/667,622, filed Jul. 3, 2024, entitled “Context-Specific Tumor-Only Mutation Classification,” the entire disclosure of which is hereby incorporated by reference herein in its entirety.

### BACKGROUND

Cancer includes an uncontrolled growth and spread of abnormal cells, e.g., tumors. The identification and classification of mutations (e.g., genetic variants relative to a reference sequence) within these tumor cells aids clinicians and researchers in understanding the underlying mechanisms of cancer development and/or guides personalized treatment strategies. For instance, mutations in cancer cells can be classified as germline mutations that are inherited from parents or somatic mutations that are acquired over time, for example, due to aging processes and/or exposures to carcinogens. While germline mutations typically occur in every cell of an individual, including normal cells and cancer cells, somatic mutations occur during the individual's lifetime and are found in a subset of cells, some of which may grow into a tumor. Because these two types of mutations arise from different biological processes, germline and somatic mutations may contribute differently to cancer development and progression and may therefore be evaluated separately. As such, classifying mutations as germline or somatic may guide treatment selection for an individual, inform on disease progression or prognosis, and/or aid discovery of new tumor drivers and therapeutic targets.

### SUMMARY

Context-specific tumor-only mutation classification is described. A mutation classification module may classify a mutation identified in sequencing data from a tumor sample as germline or somatic based on a likelihood ratio relative to a threshold, the likelihood ratio comparing a germline model likelihood of a germline model of the mutation to a somatic model likelihood of a somatic model of the mutation and the threshold calculated based on a context of the mutation. The mutation classification module may output the classification of the mutation.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ context-specific tumor-only mutation classification via a mutation classification module.

FIG. 2 depicts a simplified example of a sequencing alignment showing read coverage that supports a mutation in a sequenced tumor sample.

FIG. 3 depicts simplified example scenarios illustrating how a variant allele fraction varies based on context for germline and somatic mutations.

FIG. 4 shows an example relating sample purity to variant allele fraction for different biological contexts.

FIG. 5 depicts an example implementation of a tumor-only classification algorithm of the mutation classification module of FIG. 1 in greater detail.

FIGS. 6A-6C depict an overview of example germline and somatic mutation models that may be used by the mutation classification module of FIG. 1 to classify a mutation found in a sequenced tumor sample.

FIG. 7 depicts an illustrative example of calculating likelihoods of models given observed tumor sequencing data.

FIG. 8 depicts illustrative examples of using simulated germline and somatic log likelihood distributions to determine a threshold for classifying a mutation observed in tumor sequencing data.

FIG. 9 depicts an illustrative example of using a joint log likelihood ratio to determine a threshold for classifying a mutation observed in tumor sequencing data.

FIG. 10 depicts a workflow in an example implementation of using the mutation classification module of FIG. 1 for classifying mutations as germline or somatic.

FIG. 11 depicts an example procedure in which context-specific tumor-only mutation classification is performed.

FIG. 12 illustrates an example system including various components of an example device that can be implemented as any type of computing device as described and/or utilized with reference to FIGS. 1-11 to implement examples of the techniques described herein.

### DETAILED DESCRIPTION

### Overview

Cancer genome interpretation involves analyzing and understanding genetic alterations present within tumors, such as determined from a systematic analysis of the information encoded in deoxyribonucleic acid (DNA) sequences of tumor cells. Cancer genomes include diverse arrays of genetic variants (e.g., alterations or mutations), including point mutations, insertions/deletions (INDELs), copy number variations (CNVs), structural rearrangements, and so forth, relative to a reference genome. Cancer genome interpretation aims to systematically catalog and annotate these alterations to delineate the mutational landscape of tumors. This process helps clinicians and researchers elucidate the underlying molecular mechanisms driving tumorigenesis, identify actionable therapeutic targets, and/or predict treatment responses, for instance.

DNA sequencing technologies such as whole-genome sequencing (WGS), whole-exome sequencing (WES), and targeted sequencing panels enable cancer genomes to be profiled at nucleotide resolution, facilitating the identification of cancer-driving mutations and oncogenic pathways. As mentioned above, a mutation can be classified as germline (e.g., inherited from parents) or somatic (e.g., acquired over time by an individual). Because these two types of mutations arise from different biological mechanisms, germline mutations and somatic mutations have different roles in cancer development. Therefore, distinguishing these different mutations may be useful for studying cancer. However, distinguishing germline mutations from somatic mutations may be complex, labor-intensive, and time-consuming.

In an ideal scenario, sequencing data from a tumor sample and a normal (e.g., a non-tumor) sample from the same individual are collected. The sequencing data for the normal sample serves as a germline control against which the sequencing data for the tumor sample is compared, thus enabling somatic mutations, which are present in the tumor sample and not present in the normal sample, to be distinguished from germline mutations, which are present in the normal sample as well as the tumor sample. In other scenarios, however, unmatched sequencing is used, where the tumor sample is analyzed without a corresponding normal sample. Unmatched sequencing may be used, for example, when the normal sample is unavailable. For instance, it may be difficult to obtain the normal sample for certain types of cancers, such acute myeloid leukemia or other blood cancers, as any easily accessible non-diseased tissue may have blood cell contamination upon sampling. Cell sorting and fibroblast expansion techniques may be used to help distinguish normal cells from cancer cells but are expensive and time consuming.

When unmatched sequencing is used, mutations may be classified based on the sequencing data of the tumor sample alone. For instance, a variant allele fraction (VAF) may be used to quantify the proportion of DNA molecules in a sample that contain a particular mutation, also referred to as a variant allele, relative to the total number of DNA molecules in the sequenced sample. The VAF is typically expressed as a percentage or a fraction ranging from 0 to 1. A VAF of 0 indicates that substantially none of the DNA molecules in the sequenced sample carry the variant allele, while a VAF of 1 indicates that substantially all of the DNA molecules in the sequenced sample carry the variant allele. In the context of cancer, the VAF may provide insights into the clonality and prevalence of mutations within tumor cells. By way of example, higher VAF values may suggest that a mutation is present in a larger proportion of tumor cells in the tumor sample, whereas lower VAF values may indicate subclonal mutations or mutations present in a smaller subset of tumor cells.

However, the tumor sample often includes a mixture of cells derived from the tumor (e.g., cancerous cells) and normal, non-cancerous cells, which confounds analysis of the VAF for distinguishing between germline and somatic mutations. The VAF analysis is further hindered by copy number alteration events (e.g., duplications and deletions), which are common in cancer. For example, a same VAF value may correspond to a somatic mutation or a germline mutation depending on the particular mixture of cells in the tumor sample and copy number alteration events of a local genetic region within the tumor cells. Existing computational methods for mutation classification using the VAF typically rely on heuristic rules, statistical models, or machine learning algorithms trained on curated datasets. These approaches often lack robustness, generalizability, and scalability across diverse tumor types and sequencing platforms. As such, some germline variants may be classified as somatic mutations, and vice versa, leading to potential inaccuracies in the interpretation of the classified mutations.

To overcome these problems, context-specific tumor-only mutation classification is disclosed herein. In accordance with the described techniques, a statistical method is used to classify somatic and germline mutations from DNA sequencing data from a tumor sample without a matched control sample. In at least one implementation, the statistical method enables a mutation of interest to be classified as germline or somatic using germline mutation models and somatic mutation models that account for different mechanisms through which the mutation of interest may arise based on its context. The context, for instance, includes a purity of the tumor sample, a ploidy of the tumor sample, a copy number alteration at a local region of the mutation of interest, and a cancer cell fraction that includes the mutation of interest, as determined based on copy profile data inferred from the DNA sequencing data as a whole. The germline mutation models and the somatic mutation models may predict how the mutation of interest would be observed in the DNA sequencing data given the context, which may be compared to the actual DNA sequencing data to determine a likelihood that a given germline mutation model or somatic mutation model fits the DNA sequencing data. By way of example, the germline mutation models and the somatic mutation models predict VAFs for the mutation of interest. A highest likelihood germline mutation model (e.g., of the germline mutation models) and a highest likelihood somatic mutation model (e.g., of the somatic mutation models) may be compared using a likelihood ratio, which indicates whether the highest likelihood germline mutation model or the highest likelihood somatic mutation model better fits the data. Moreover, in one or more implementations, joint evidence is used from multiple tumor samples from the same patient in order to increase sensitivity and/or decrease a false positive rate.

In at least one implementation, a logarithm of the likelihood ratio (e.g., a log likelihood ratio) is compared to a context-based threshold. The mutation of interest may be classified as somatic in response to the log likelihood ratio being less than the context-based threshold or classified as germline in response to the log likelihood ratio being greater than the context-based threshold. The context-based threshold may be calculated and/or adjusted based on the mutation of interest rather than being a single threshold used to classify all mutations or a machine learning-trained threshold that is not generalizable across samples and/or sequencing platforms.

In this way, mutations of a tumor sample may be distinguished as germline or somatic with higher accuracy and interpretability in tumor-only samples. Moreover, the statistical method described herein is broadly applicable to a variety of sequencing techniques and tumor sample types. As a result, disease driver discovery is increased, which enhances the identification of potential therapeutic targets.

In some aspects, the techniques described herein relate to a system for context-specific mutation classification, including: a mutation classification module implemented in a non-transitory computer-readable storage medium and configured to: classify a mutation identified in sequencing data from a tumor sample as germline or somatic based on a likelihood ratio relative to a threshold that is calculated based on a context of the mutation, the likelihood ratio comparing a germline model likelihood of a germline model of the mutation to a somatic model likelihood of a somatic model of the mutation; and output the classification of the mutation.

In some aspects, the techniques described herein relate to a system, wherein the germline model is selected from a plurality of germline models based on a fit of the germline model to the sequencing data relative to other germline models of the plurality of germline models, and the somatic model is selected from a plurality of somatic models based on a fit of the somatic model to the sequencing data relative to other somatic models of the plurality of somatic models.

In some aspects, the techniques described herein relate to a system, wherein the mutation classification module is further configured to: generate a likelihood distribution of a true measurement of alternate counts for the mutation based on the sequencing data; determine expected variant allele fractions of the mutation for the plurality of germline models and the plurality of somatic models based on the context of the mutation; determine respective fits of the plurality of germline models and the plurality of somatic models to the likelihood distribution based on the expected variant allele fractions; select the germline model based on the respective fits of the plurality of germline models; and select the somatic model based on the respective fits of the plurality of somatic models.

In some aspects, the techniques described herein relate to a system, wherein the likelihood distribution is generated using a beta binomial distribution.

In some aspects, the techniques described herein relate to a system, wherein the mutation classification module is further configured to: determine the context of the mutation based on copy number data of the sequencing data, the context including a purity of the tumor sample and a ploidy of the tumor sample.

In some aspects, the techniques described herein relate to a system, wherein, to determine the context of the mutation based on the copy number data of the sequencing data, the mutation classification module is configured to: generate candidate copy profile interpretations including different values for the purity of the tumor sample and the ploidy of the tumor sample based on the copy number data; and select a copy profile interpretation of the candidate copy profile interpretations based on a fit of the copy profile interpretation to the copy number data, wherein the purity of the tumor sample corresponds to a purity value of the selected copy profile interpretation and the ploidy of the tumor sample corresponds to a ploidy value of the selected copy profile interpretation.

In some aspects, the techniques described herein relate to a system, wherein the context further includes a copy number alteration at a genetic location of the mutation, a first cancer cell fraction that includes the copy number alteration, and a second cancer cell fraction that includes the mutation, and wherein the mutation classification module is further configured to: infer each of the copy number alteration, the first cancer cell fraction, and the second cancer cell fraction based on the purity of the tumor sample, the ploidy of the tumor sample, and the copy number data.

In some aspects, the techniques described herein relate to a system, wherein to classify the mutation, the mutation classification module is configured to: classify the mutation as somatic in response to a logarithm of the likelihood ratio being less than the threshold; or classify the mutation as germline in response to the logarithm of the likelihood ratio being greater than or equal to the threshold.

In some aspects, the techniques described herein relate to a system, wherein the germline model likelihood is a sum of germline model likelihood distributions determined for a plurality of tumor samples from a same subject, the plurality of tumor samples including the tumor sample, and the somatic model likelihood is a sum of somatic model likelihood distributions determined for the mutation from the plurality of tumor samples.

In some aspects, the techniques described herein relate to a system, wherein the mutation classification module is further configured to: calculate the threshold based on the context of the mutation and further based on a desired performance metric and the somatic model of the mutation or the germline model of the mutation.

In some aspects, the techniques described herein relate to a system, wherein: the threshold is calculated based on the somatic model of the mutation in response to the desired performance metric being a target sensitivity for classifying somatic mutations as somatic; or the threshold is calculated based on the germline model of the mutation in response to the desired performance metric being a target false positive rate for classifying germline mutations as somatic.

In some aspects, the techniques described herein relate to a method for context-specific mutation classification, including: receiving a sequencing alignment for a tumor sample, the sequencing alignment including a plurality of sequencing reads aligned to a reference sequence; identifying a mutation at a genetic region where at least a subset of the plurality of sequencing reads differs from the reference sequence; classifying the mutation as germline or somatic based on a log likelihood ratio relative to a threshold, the log likelihood ratio indicating a relative fit of a germline mutation model of the mutation and a somatic mutation model of the mutation to data from the sequencing alignment based on a context of the mutation; and outputting the classification of the mutation.

In some aspects, the techniques described herein relate to a method, further including: calculating the threshold based on the context of the mutation and further based on one of a desired sensitivity for classifying somatic mutations as somatic or a desired false positive rate for classifying germline mutations as somatic.

In some aspects, the techniques described herein relate to a method, wherein the context includes a purity of the tumor sample, a ploidy of the tumor sample, a copy number variation at the genetic region, a first fraction of cancer cells in the tumor sample that includes the copy number variation, and a second fraction of cancer cells in the tumor sample that includes the mutation.

In some aspects, the techniques described herein relate to a method, further including: selecting the germline mutation model from a set of germline mutation models based on a germline model likelihood of the germline mutation model relative to other germline mutation models of the set of germline mutation models; and selecting the somatic mutation model from a set of somatic mutation models based on a somatic model likelihood of the somatic mutation model relative to other somatic mutation models of the set of somatic mutation models.

In some aspects, the techniques described herein relate to a method, wherein selecting the germline mutation model from the set of germline mutation models further includes: computing germline model likelihoods for respective germline mutation models based on respective expected variant allele fractions for the respective germline mutation models and the data from the sequencing alignment; and selecting the germline mutation model having a greatest germline model likelihood of the germline model likelihoods.

In some aspects, the techniques described herein relate to a method, wherein selecting the somatic mutation model from the set of somatic mutation models further includes: computing somatic model likelihoods for respective somatic mutation models based on respective expected variant allele fractions of the respective somatic mutation models and the data from the sequencing alignment; and selecting the somatic mutation model having a greatest somatic model likelihood of the somatic model likelihoods.

In some aspects, the techniques described herein relate to a method for context-specific mutation classification, including: receiving sequencing alignments for a plurality of tumor samples obtained from an individual, the sequencing alignments including a plurality of sequencing reads aligned to a reference sequence for individual tumor samples of the plurality of tumor samples; identifying a mutation at a genetic region where at least a subset of the plurality of sequencing reads differs from the reference sequence for the plurality of tumor samples; separately calculating log likelihood ratio distributions for individual samples of the plurality of tumor samples, each log likelihood ratio distribution indicating a relative fit of a germline model of the mutation and a somatic model of the mutation to data from a corresponding sequencing alignment; classifying the mutation as germline or somatic based on a joint log likelihood ratio for the plurality of tumor samples relative to a joint threshold, the joint log likelihood ratio being a sum of the log likelihood ratio distributions of the individual samples; and outputting the classification of the mutation.

In some aspects, the techniques described herein relate to a method, further including calculating the joint threshold based on a context of the mutation and a desired performance metric for classifying the mutation, and wherein classifying the mutation as germline or somatic based on the joint log likelihood ratio relative to the joint threshold includes: classifying the mutation as germline in response to the joint log likelihood ratio being greater than or equal to the joint threshold; or classifying the mutation as somatic in response to the joint log likelihood ratio being less than the joint threshold.

In some aspects, the techniques described herein relate to a method, wherein: calculating the joint threshold is further based on a sum of germline log likelihood ratio distributions for the plurality of tumor samples in response to the desired performance metric being a desired false positive rate for classifying germline mutations as somatic; or calculating the joint threshold is further based on a sum of somatic log likelihood ratio distributions for the plurality of tumor samples in response to the desired performance metric being a desired true positive rate for classifying somatic mutations as somatic.

In the following discussion, an example environment is first described that may employ the techniques described herein. Example implementation details and procedures are then described which may be performed in the example environment as well as other environments. Consequently, performance of the example procedures is not limited to the example environment and the example environment is not limited to performance of the example procedures.

### Example Environment

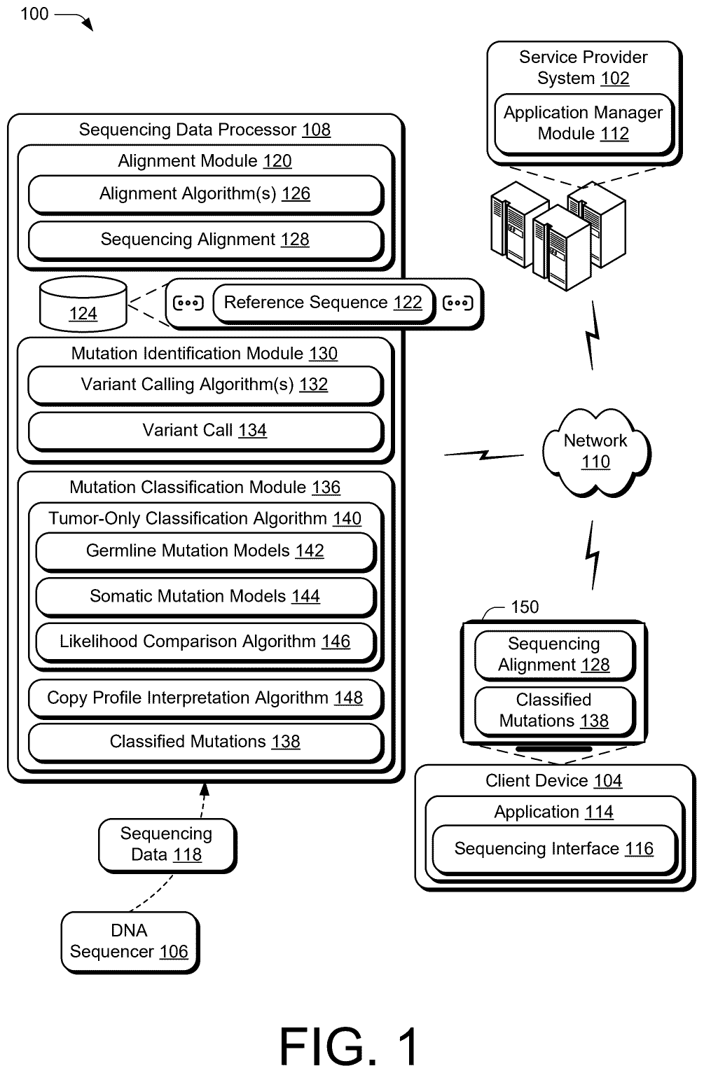

FIG. 1 is an illustration of an environment 100 in an example implementation that is operable to employ context-specific tumor-only mutation classification as described herein. The illustrated environment 100 includes a service provider system 102, a client device 104, a DNA sequencer 106, and a sequencing data processor 108 that are communicatively coupled, one to another, via a network 110. Although the sequencing data processor 108 is illustrated as separate from the service provider system 102, the client device 104, and the DNA sequencer 106, this functionality may be incorporated as part of the service provider system 102, the client device 104, and/or the DNA sequencer 106, further divided among other entities, and so forth. By way of example, an entirety of or portions of the functionality of the sequencing data processor 108 may be incorporated as part of the DNA sequencer 106. Additionally, or alternatively, an entirety of or portions of the client device 104 may be incorporated as part of the DNA sequencer 106.

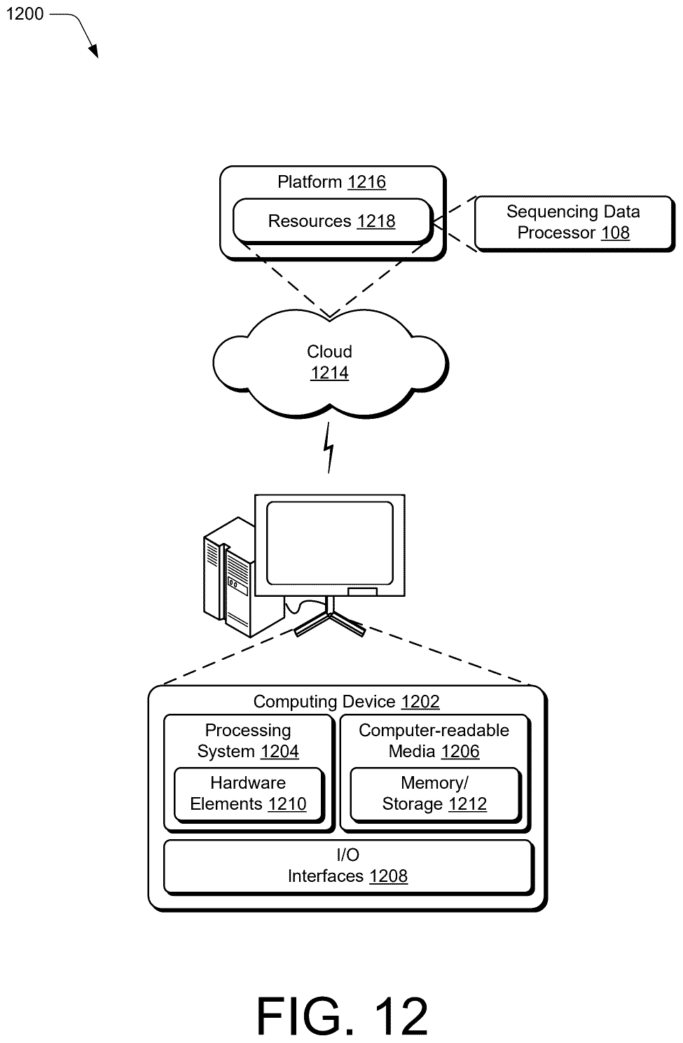

Computing devices that are usable to implement the service provider system 102, the client device 104, and the sequencing data processor 108 may be configured in a variety of ways. A computing device, for instance, may be configured as a desktop computer, a laptop computer, a mobile device (e.g., assuming a handheld configuration such as a tablet or mobile phone), and so forth. Thus, the computing device may range from full resource devices with substantial memory and processor resources (e.g., personal computers, game consoles) to a low-resource device with limited memory and/or processing resources (e.g., mobile devices). Additionally, a computing device may be representative of a plurality of different devices, such as multiple servers utilized to perform operations “over the cloud,” as further described in relation to FIG. 12.

The service provider system 102 is illustrated as including an application manager module 112 that is representative of the functionality to provide access to the sequencing data processor 108 to a user of the client device 104 via the network 110. The application manager module 112, for instance, may expose content or functionality of the sequencing data processor 108 that is accessible via the network 110 by an application 114 of the client device 104. The application 114 may be configured as a network-enabled application, a browser, a native application, and so on, that exchanges data with the service provider system 102 via the network 110. The data can be employed by the application 114 to enable the user of the client device 104 to communicate with the service provider system 102, such as to receive application updates and features when the service provider system 102 provides functionality to manage the application 114.

In the context of the described techniques, the application 114 includes the functionality to input parameters for a sequencing event as well as to analyze data generated by the sequencing event. In the illustrated example, the application 114 includes a sequencing interface 116 that is implemented at least partially in hardware of the client device 104 for facilitating communication between the client device 104 and the sequencing data processor 108. By way of example, the sequencing interface 116 includes functionality to receive inputs to the sequencing data processor 108 from the client device 104 (e.g., from a user of the client device 104) and output information, data, and so forth from the sequencing data processor 108 to the client device 104, as will be further elaborated herein.

The sequencing event includes determining an order of nucleotides (e.g., adenine, thymine or uracil, cytosine, and guanine) in a sample of nucleic acids, such as derived from a biological sample. The order of nucleotides is referred to herein as a “sequence.” The nucleotides are also referred to as “bases.” The sequencing event will be described herein with respect to deoxyribonucleic acid (DNA) sequencing (e.g., whole-exome, whole-genome, or targeted panel sequencing).

The DNA sequencer 106 is configured to produce sequencing data 118 that is analyzed by the sequencing data processor 108 to determine the order of nucleotides in a sample. In at least one implementation, the sequencing data 118 comprise a text-based file format, such as FASTQ files that store both nucleotide sequence information and quality scores for the bases in a sequencing read. In variations, the sequencing data 118 comprise another type of file format. The DNA sequencer 106 may use one of a plurality of sequencing techniques to produce the sequencing data 118. By way of example, the DNA sequencer 106 may use a short read sequencing technique that produces sequence fragments typically ranging from approximately 10 bases to approximately 1000 bases and more typically from approximately 50 bases to approximately 500 bases. Sequence fragments produced via short read sequencing techniques are also referred to herein as “short reads.” Long read sequencing techniques produce sequence fragments that typically range from 1000 bases to 1,000,000 bases and more typically from 5000 bases to 500,000 bases in length. Sequence fragments produced via long read sequencing techniques are also referred to herein as “long reads.” The DNA sequencer 106 may be configured for whole-genome sequencing, where both protein-coding regions and non-coding regions are sequenced, or whole-exome sequencing, where only protein-coding regions (e.g., exons) are sequenced, for example.

Regardless of the sequencing technique, the sequencing data processor 108 receives the sequencing data 118 and determines a sequence (e.g., a consensus sequence) of the nucleotides in the sample therefrom based at least in part on an output of an alignment module 120. The alignment module 120 is representative of functionality for performing read alignment of the sequencing data 118. Read alignment, also referred to simply as “alignment,” involves mapping the sequence fragments (e.g., long reads and/or short reads) to locations in the genome using a reference sequence 122. The reference sequence 122 may be selected from a variety of nucleic acid sequences against which a sequence of a sample can be compared for determining an order of the nucleotides in the sample as well as determining variants of the sequence of the sample, as will be elaborated below. The reference sequence 122 is a reference genome or a portion thereof. In one or more implementations, the sequencing data processor 108 includes or otherwise accesses a storage device 124 storing the reference sequence 122. The storage device 124 may store one or more other reference sequences in addition to the reference sequence 122, as indicated by ellipses in FIG. 1. By way of example, different reference sequences may correspond to different sample types or may come from a pangenome, which includes several high-quality, curated assemblies of individual genomes that may be represented jointly as a graph. As such, the reference sequence 122 is selected based on its similarity to the sample evaluated via the sequencing event, at least in some implementations. Moreover, the reference sequence 122 may include a combination of more than one individual reference sequence. By way of example, the reference sequence 122 may be a curated representation of an average population-level genome of an organism (e.g., the average human genome) or a portion of the average population-level genome.

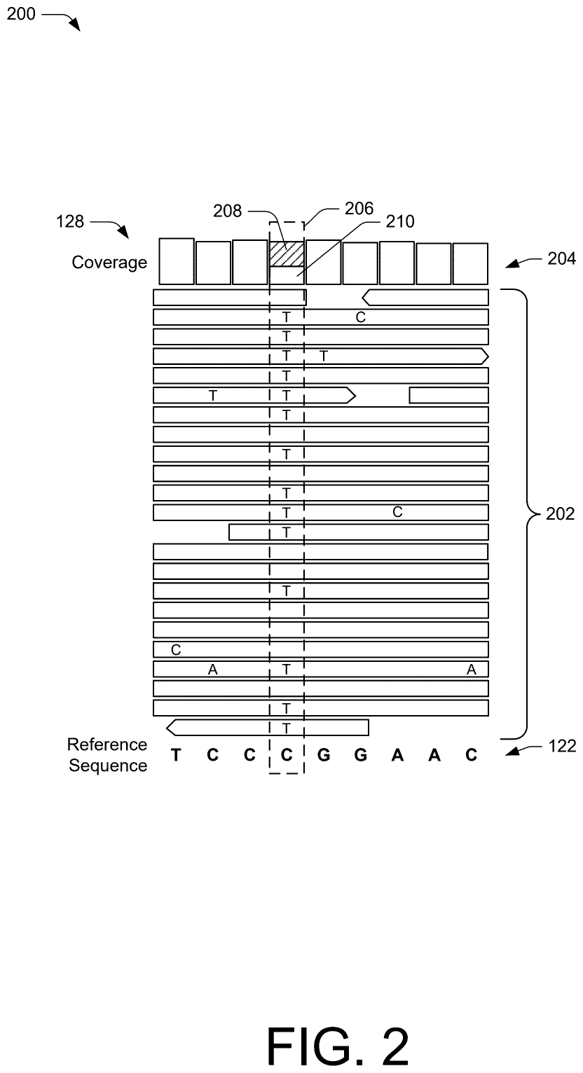

In at least one implementation, the alignment module 120 is configured to map the sequencing data 118 to the reference sequence 122 via one or more alignment algorithms 126 to generate a sequencing alignment 128, also referred to herein as aligned reads. The one or more alignment algorithms 126 include functionality for finding an alignment that increases (e.g., maximizes) a similarity between a read and the reference sequence 122 using a scoring system that considers possible mismatches between the reference sequence 122 and the sequencing data 118, e.g., insertions, deletions, and point substitutions. The sequencing alignment 128 comprises sequence fragments (e.g., reads) that have been successfully mapped to the reference sequence 122, such as illustrated with respect to FIG. 2 and further described herein.

In the context of determining a genome of an individual, the sequencing alignment 128 may be used to determine the consensus sequence of the nucleotides in the sample. By way of example, at respective positions in the sequencing alignment 128, the nucleotide present in the majority of read sequences may be chosen for the consensus sequence at that position. This process may involve counting the occurrences of each base at a specific position, which may be the same as or different than the reference sequence 122. In the context of cancer genomics, however, the sequencing alignment 128 may be used to quantify a proportion of nucleic acid molecules in a sample (e.g., a tumor sample) that include a particular genetic variant, such as mutation or polymorphism. This quantity is referred to as a variant allele fraction (VAF), which will be further described herein.

“Variant calling” refers to the identification and/or characterization of these genetic variants in a sequence determined for a sample (e.g., a tumor sample) when compared to the reference sequence 122 (e.g., a reference exome). These variants may include short variants, such as single nucleotide polymorphisms (SNPs, where one nucleotide is changed to another at a specific position in the genome), insertions (e.g., the addition of one or more nucleotides at a specific position in the genome), and deletions (e.g., the removal of one or more nucleotides at a specific position in the genome). Insertions and deletions may be collectively referred to as “INDELs.” Additionally or alternatively, the variants may include larger, structural variations, such as copy number variants (CNVs, where a segment of DNA ranging from kilobases to megabases in size is duplicated or deleted), inversions (e.g., where a segment of DNA is reversed in orientation), INDELs involving larger segments (e.g., more than 50 nucleotides), translocations (e.g., where a segment of DNA is moved from one location to another, often involving the exchange of genetic material between non-homologous chromosomes), and replacements (e.g., where a segment of DNA is replaced or substituted by another, which may include additional changes such as insertions, duplications, or other rearrangements).

Accordingly, in at least one implementation, the sequencing data processor 108 includes a mutation identification module 130 representative of the functionality for determining genomic differences between the sequencing alignment 128 and the reference sequence 122. The mutation identification module 130 includes one or more variant calling algorithms 132 to generate a variant call 134 using statistical and/or computational methods. By way of example, the one or more variant calling algorithms 132 may analyze the sequencing alignment 128 to detect positions that differ from the reference sequence 122, thus indicating potential variants. In at least one implementation, the one or more variant calling algorithms 132 consider factors such as read depth (e.g., coverage), base quality scores of the sequencing data 118, a mapping quality of the sequencing alignment 128, and strand bias to balance sensitivity (the ability to detect true variants) and specificity (the ability to avoid false positives). The variant call 134 includes an indication of one or more variants, such as variant alleles, that are determined to be present in the sequencing alignment 128 compared to the reference sequence 122.

In general, the terms “mutation” and “variant” as used herein refer to any observed deviation (e.g., variation) from a reference (e.g., the reference sequence 122). For example, mutations that cause genetic variants can arise from biological processes such as natural variation in the population, aging processes, DNA damage from environmental exposures, and so forth. Germline variants or mutations, also referred to herein as “germline events,” are mutations that are inherited directly from an individual's parents and are typically present in every cell in the individual's body. Somatic variants or mutations, also referred to herein as “somatic events,” are acquired over the individual's lifetime and are typically found in a subset of cells or tissues. While both germline and somatic mutations may contribute to the development of cancer, they arise from different mechanisms. As such, in order to gain a better understanding of biological processes that drive cancer, it is desirable to distinguish between somatic mutations and germline mutations in the sequencing data 118 obtained from a tumor sample.

Thus, in accordance with the techniques described herein, the sequencing data processor 108 includes a mutation classification module 136. The mutation classification module 136 is representative of the functionality to distinguish between somatic mutations and germline mutations, resulting in classified mutations 138. In some scenarios, matched sequencing (e.g., matched whole-exome sequencing) is used in which a tumor sample and a normal (e.g., non-tumor) sample are obtained from the same individual. Although referred to as a tumor sample (or tumor cell sample), the tumor sample often includes a mixture of cells derived from the tumor (e.g., cancerous cells) and normal, non-cancerous cells. When matched sequencing is used, the sequencing data 118 may be obtained for both the tumor sample and the normal sample, and the sequencing data 118 for the normal sample may serve as a germline control against which the sequencing data 118 for the tumor sample are compared. This enables the mutation classification module 136 to distinguish the somatic mutations, which are present in the tumor sample and not present in the normal sample, from the germline mutations, which are present in the normal sample as well as the tumor sample, in the classified mutations 138. In other scenarios, however, unmatched sequencing is used, where the tumor sample is analyzed without a corresponding normal sample. Unmatched sequencing may be used when the normal sample is unavailable. When unmatched sequencing is used, somatic mutations are identified based on the sequencing data 118 of the tumor sample itself, such as based on the VAF of a given mutation. However, using existing heuristic, empirically trained techniques, some germline variants may be mistakenly classified as somatic mutations, and vice versa, leading to potential inaccuracies in the interpretation of the classified mutations 138.

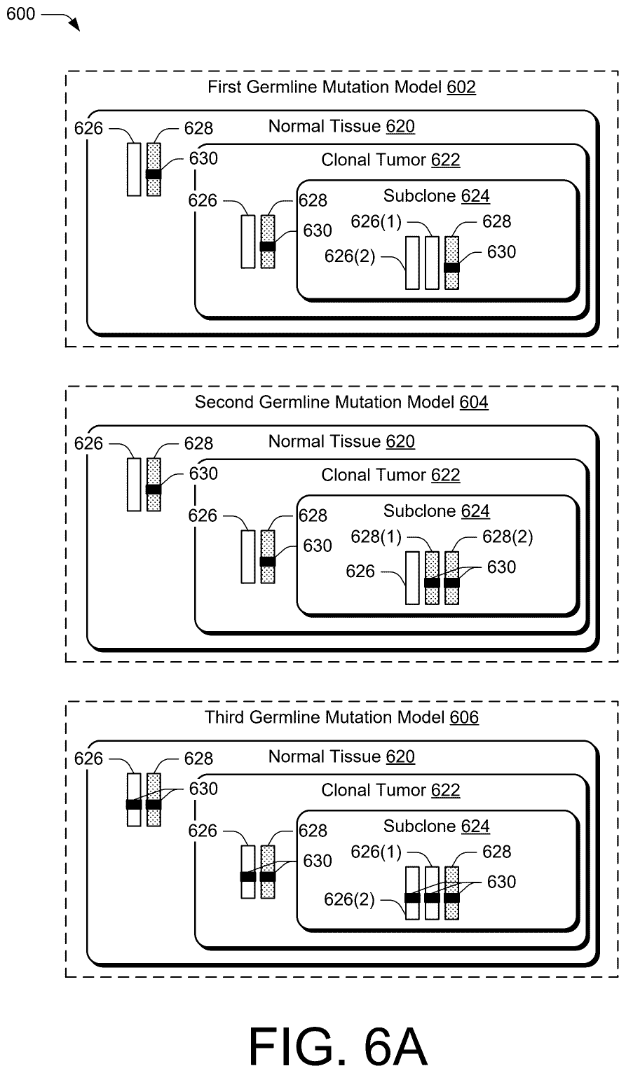

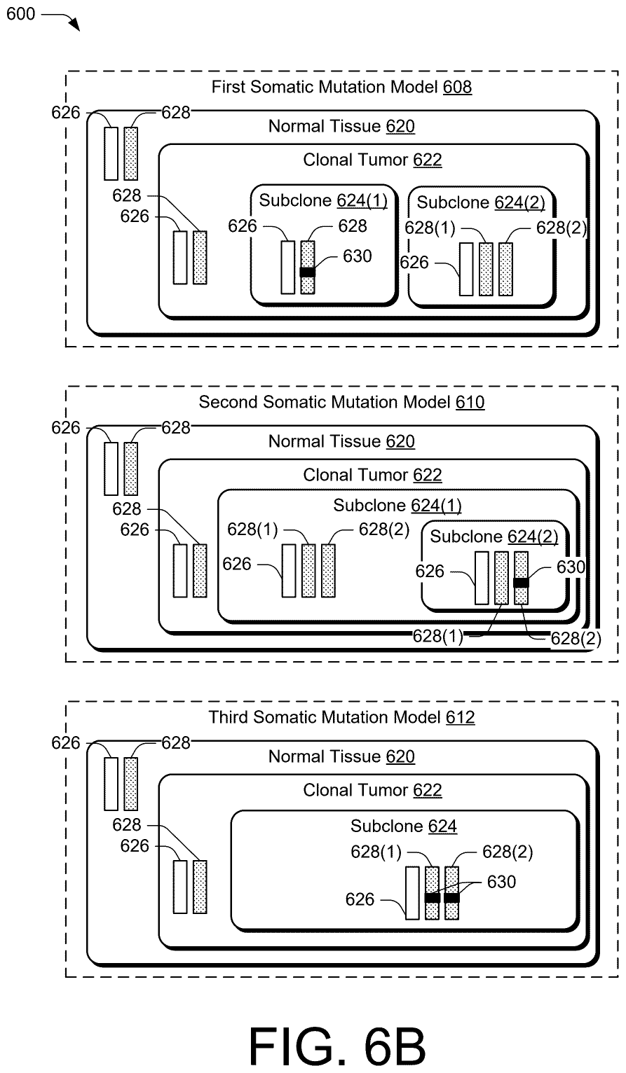

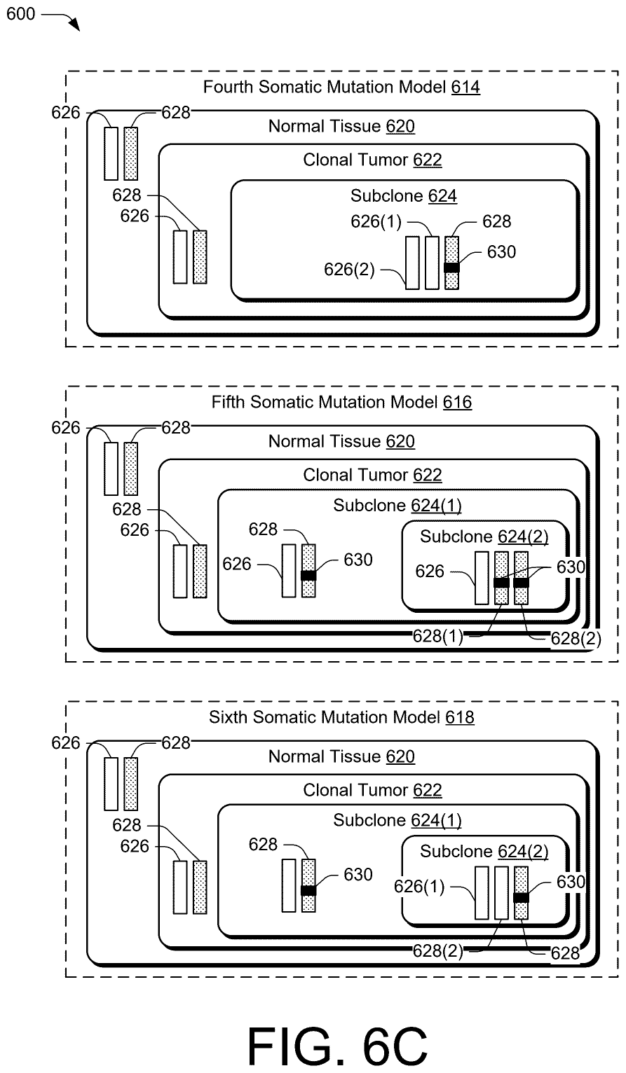

In order to overcome the mutation classification issues related to tumor-only, unmatched sequencing, the mutation classification module 136 includes a tumor-only classification algorithm 140. In at least one implementation, the tumor-only classification algorithm 140 is a statistical method to classify somatic and germline mutations from sequencing data 118 derived from tumors generally, but particularly from tumors lacking corresponding germline control samples. By way of example, the tumor-only classification algorithm 140 estimates an expected read support (e.g., a number or proportion of sequencing reads that provide evidence for a specific genetic variant at a particular genomic position) for a somatic event versus a germline event and calculates a likelihood ratio based on observed read support in the sequencing alignment 128. As a part of this, the tumor-only classification algorithm 140 uses germline mutation models 142 and somatic mutation models 144 to predict how a germline or somatic mutation, respectively, will be observed in the sequencing alignment 128 and executes a likelihood comparison algorithm 146 given the actual sequencing alignment 128 to determine which model (e.g., a somatic model or a germline model) is more likely to produce the observed read support. By way of example, the germline mutation models 142 are built to model how germline mutations manifest in tumor cells and normal cells, and the somatic mutation models 144 are built to model how somatic mutations occur in tumor cells. Examples of the germline mutation models 142 and the somatic mutation models 144 will be further described herein, e.g., with respect to FIGS. 6A-6C.

In at least one implementation, the tumor-only classification algorithm 140 generates expected VAFs of a mutation of interest for the germline mutation models 142 and the somatic mutation models 144. Because the VAF of the mutation varies based on a context of the mutation in terms of, for example, a purity of the tumor sample, a ploidy of the tumor sample, a copy number alteration at a local region of the mutation of interest, a cancer cell fraction that includes the copy number alteration, and a cancer cell fraction that includes the mutation of interest, in one or more implementations, the tumor-only classification algorithm 140 receives input from a copy profile interpretation algorithm 148. The copy profile interpretation algorithm 148 is configured to rescale the sequencing data 118 to DNA originating from the tumor cells, rather than the mixture of tumor cells and normal cells in the tumor sample. In at least one implementation, the copy profile interpretation algorithm 148 estimates the purity, the ploidy, and/or the cancer cell fraction (CCF) of the sequence tumor sample. The purity refers to a percentage (e.g., ranging from 0% to 100%) or proportion (e.g., ranging from 0 to 1) of the cells in the sequenced tumor sample that actually came from tumor cells. The ploidy refers to an average number of copies of the genome in the tumor cells, as this may deviate from the diploid nature of normal cells due to amplification or deletion. The CCF refers to a fraction of tumor cells that contain a somatic event (such as a mutation or copy number variant).

The purity, the ploidy, and the CCF may collectively comprise a copy profile of the tumor cell sample. By way of example, the copy profile interpretation algorithm 148 determines the purity, the ploidy, and/or the CCF based at least in part on how candidate copy profile interpretations (e.g., different values for purity, ploidy, and/or CCF) fit observed data of the sequencing alignment 128. Thus, the copy profile interpretation algorithm 148 provides the mutation context used by the germline mutation models 142 and the somatic mutation models 144 in determining the expected VAFs.

The client device 104 is shown displaying, via a display device 150, the sequencing alignment 128, or a portion thereof, as well as the classified mutations 138. By way of example, the display device 150 may display a portion of sequencing alignment 128 as a string of characters representing the sequence of nucleotides in the portion. Additionally, or alternatively, the display device 150 may display the sequencing alignment 128 as a visual representation of the reads aligned with the reference sequence 122 along with an indication of a nucleotide identified at a specific location. The classified mutations 138 may be displayed by the display device 150 as a visual representation of genomic location(s) where germline and/or somatic variant(s) are present and/or as a list of detected germline and/or somatic variant(s) and their genomic location(s). It is to be appreciated that the classified mutations 138 and the sequencing alignment 128 are also stored in memory, in a single data file or multiple data files, for subsequent access.

In this way, the mutation classification module 136, via the tumor-only classification algorithm 140, generates the classified mutations 138 for unmatched sequencing with increased accuracy. Accordingly, the mutations found in certain diseases, such as acute myeloid leukemia and other blood cancers, may be identified and classified according to their lineage without relying on expensive and time-consuming cell sorting or techniques that produce inaccurate and/or unreliable results. As a result, disease driver discovery is increased, which enhances the identification of potential therapeutic targets.

Before describing additional details of example implementations of the mutation classification module 136, examples scenarios will now be described in order to put the sequencing data, the VAF, and the difference between germline and somatic mutations into context.

FIG. 2 depicts a simplified example 200 of the sequencing alignment 128 showing read coverage that supports a mutation in a sequenced tumor sample. The example 200 includes reads 202, which are aligned to the reference sequence 122. Letters in the reads 202 indicate a position where a given read 202 has a nucleotide that deviates from the reference sequence 122. The example 200 also depicts coverage 204 as a bar plot. The coverage 204 refers to the number of observed reads that align to a particular region of the reference sequence 122. A genetic region 206 includes an alteration in the sequence for a plurality of the reads 202 compared to the reference sequence 122. In the example 200, the plurality of the reads 202 at the genetic region 206 include a “T” rather than the reference sequence 122 base “C.”

For a region of interest, an alternate count refers to the number of reads containing an alteration in their sequences (e.g., relative to the reference sequence 122) that supports a mutation. In contrast, a reference count refers to the number of reads at the region of interest that include the reference base. In the example depicted in FIG. 2, the genetic region 206 includes an alternate count 208 and a reference count 210. The alternate count 208 quantifies the number of reads 202 having “T” at the genetic region 206, whereas the reference count 210 quantifies the number of reads 202 having “C” at the genetic region 206.

In at least one implementation, the alternate count 208 is used to calculate the VAF of the genetic region 206, which represents the proportion of the reads 202 that support the “T” mutation. By way of example, the VAF of the genetic region 206 may be calculated as the alternate count 208 divided by the total coverage (e.g., the sum of the alternate count 208 and the reference count 210 in this example). Thus, the VAF refers to the proportion (e.g., fraction) of the reads 202 that support a variant allele. However, as will be elaborated below, the VAF alone does not indicate whether the variant allele is the result of a germline event or a somatic event.

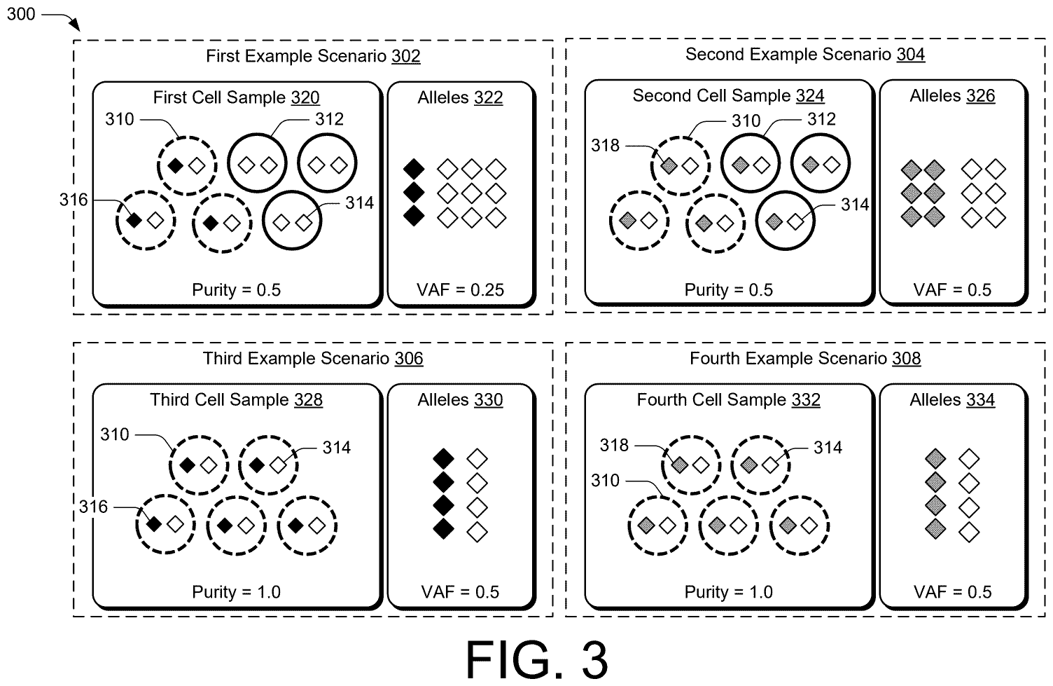

FIG. 3 depicts simplified example scenarios 300 to illustrate how the variant allele fraction varies based on context for germline and somatic mutations. The simplified example scenarios 300 include a first example scenario 302, a second example scenario 304, a third example scenario 306, and a fourth example scenario 308. Tumor cells 310 are depicted as dashed circles, and normal cells 312 (e.g., non-cancerous, or healthy, cells) are depicted as solid circles. For illustrative clarity, only a portion of the tumor cells 310 and the normal cells 312 are labeled. The simplified example scenarios 300 do not include copy number events. That is, the alleles are not duplicated or deleted, as is the case with copy number alterations or variants. As such, the tumor cells 310 and the normal cells 312 each include two alleles, represented as diamonds in FIG. 3. A wild-type allele 314 is depicted as an unfilled (e.g., white-filled) diamond, a somatic mutation 316 is depicted as a black-filled diamond, and a germline mutation 318 is depicted as a shaded diamond. Only a portion of the wild-type allele 314, the somatic mutation 316, and the germline mutation 318 are labeled for illustrative clarity. As mentioned above, somatic mutations are those that occur during an organism's lifetime and are typically found in a subset of cells or tissues, and germline mutations are those that are inherited and typically occur in every or almost every cell and tissue.

The first example scenario 302 includes a first cell sample 320 that is a mixture of the tumor cells 310 and the normal cells 312. In particular, the first cell sample 320 includes three tumor cells 310 and three normal cells 312, giving the first cell sample 320 a purity of 0.5 (or 50%) because half of the cells in the first cell sample 320 are tumor cells. In the first example scenario 302, the normal cells 312 do not include mutational variants, and thus only include the wild-type allele 314. That is, both alleles of the normal cells 312 are the wild-type allele 314. The tumor cells 310 include the somatic mutation 316 on one allele and one wild-type allele 314. This results in alleles 322 of the first cell sample 320 having a variant allele fraction (VAF) of 0.25. That is, a quarter of the alleles 322 of the first cell sample 320 are the somatic mutation 316.

The second example scenario 304 includes a second cell sample 324 that is also a mixture of the tumor cells 310 and the normal cells 312. Similar to the first cell sample 320 of the first example scenario 302, the second cell sample 324 of the second example scenario 304 includes three tumor cells 310 and three normal cells 312, giving the first cell sample 320 a purity of 0.5. However, unlike the first cell sample 320, the second cell sample 324 includes the germline mutation 318. The germline mutation 318 is present in every cell of the second cell sample 324, e.g., the tumor cells 310 and the normal cells 312. The wild-type allele 314 is also present in the tumor cells 310 and the normal cells 312. This results in alleles 326 of the second cell sample 324 having a VAF of 0.5. That is, half of the alleles 326 of the second cell sample 324 have the germline mutation 318.

In comparing the first example scenario 302 and the second example scenario 304, it is possible to distinguish between the somatic mutation 316 and the germline mutation 318 based on the VAF. For example, the inclusion of the normal cells 312 in the first cell sample 320 and the second cell sample 324, and thus the decrease in purity to 50% in both samples, enables the VAF to be used to distinguish between germline and somatic mutations.

The third example scenario 306 includes a third cell sample 328 that includes the tumor cells 310 and no normal cells 312. Because the third cell sample 328 includes only the tumor cells 310, the purity of the third cell sample 328 is 1.0 (or 100%). The tumor cells 310 include the somatic mutation 316 along with the wild-type allele 314, resulting in alleles 330 of the third cell sample 328 having a VAF of 0.5. That is, half of the alleles 330 of the third cell sample 328 have the somatic mutation 316.

The fourth example scenario 308 includes a fourth cell sample 332. Similar to the third cell sample 328, the fourth cell sample 332 includes the tumor cells 310 and no normal cells 312, giving the fourth cell sample 332 a purity of 1.0. However, unlike the third cell sample 328, the tumor cells 310 in the fourth cell sample 332 include the germline mutation 318 on one allele. This results in alleles 334 of the fourth cell sample 332 having a VAF of 0.5.

In comparing the third example scenario 306 and the fourth example scenario 308, it is not possible to distinguish between the somatic mutation 316 and the germline mutation 318 based on the VAF, as they are both 0.5.

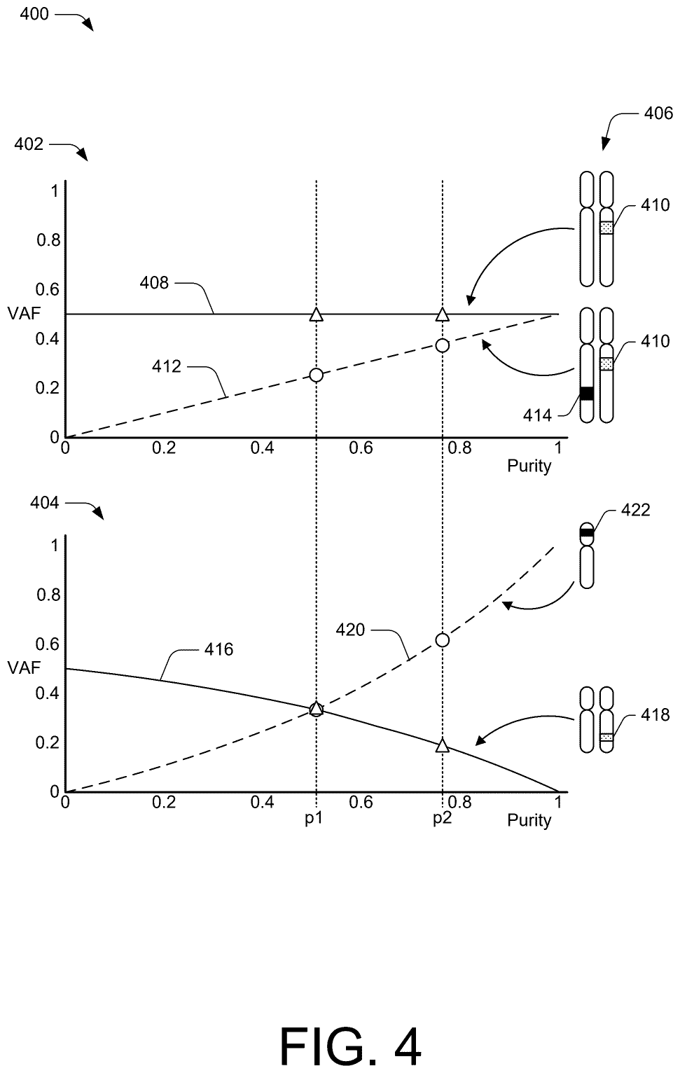

The separation versus similarity between germline and somatic mutations with respect to purity and VAF in different contexts will now be further described. FIG. 4 shows an example 400 relating sample purity to variant allele fraction for different biological contexts. The example 400 includes a first graph 402 and a second graph 404 of purity (horizontal axes) versus VAF (vertical axes). In particular, the first graph 402 corresponds to a diploid context, where there are two copies of an allele in tumor cells. The second graph 404 corresponds to a clonal deletion of the germline allele in the tumor cells, which is further illustrated in accompanying chromosomal diagrams 406. A purity of zero refers to a sample where no tumor cells are present. A purity of one refers to a sample where no normal cells are present. As such, a purity of 0.5, for instance, represents a sample comprising approximately equal quantities of normal cells and tumor cells.

Referring first to the first graph 402, a germline plot 408 corresponding to a germline mutation 410 is a flat line (e.g., having a slope of zero) at a VAF of 0.5. For example, because the germline mutation 410 is an inherited mutation, the VAF does not change with respect to purity. Instead, both normal cells and tumor cells include the germline mutation 410 in one allele of the diploid pair of chromosomes, as illustrated in the chromosomal diagrams 406. In contrast, a dashed somatic plot 412 of a somatic mutation 414 increases linearly as the purity increases. For example, because the somatic mutation 414 is present in all of the tumor cells in this scenario (CCF=1), and not normal cells, the VAF is zero when no tumor cells are included in the sample (e.g., when the purity is zero) and 0.5 when no normal cells are included in the sample (e.g., when the purity is one).

Referring to the second graph 404, a germline plot 416 of a germline mutation 418 shows the VAF decreasing non-linearly as the purity increases, whereas a dashed somatic plot 420 representing a somatic mutation 422 increases non-linearly as the purity increases. The second graph 404 represents a scenario where the allele with the germline mutation 418 undergoes clonal deletion in the tumor cells. As such, the VAF of the germline plot 416 is maximal (e.g., equal to 0.5) when no tumor cells are present in the sample and minimal (e.g., equal to zero) when no normal cells are present in the sample. In contrast, due to the deletion of the allele having the germline mutation 418 in the tumor cells, the VAF of the dashed somatic plot 420 increases to one at a purity of one. For instance, when only the tumor cells are present, the remaining chromosome copy carries the somatic mutation 422, as illustrated in the chromosomal diagrams 406.

As can be appreciated by comparing the first graph 402 and the second graph 404, a purity where somatic and germline VAFs are different in diploid regions may be similar in certain aneuploid regions, and vice versa. By way of example, at a purity p1 (e.g., a purity of 0.5), there is significant separation between the germline plot 408 and the dashed somatic plot 412 of the first graph 402. In the diploid scenario of the first graph 402, at the purity p1, for instance, the germline plot 408 has a VAF of 0.5, whereas the dashed somatic plot 412 has a VAF of 0.25. This separation is smaller at a purity p2 (e.g., a purity of 0.75). For instance, the VAF of the germline plot 408 remains at 0.5, while the VAF of the dashed somatic plot 412 increases to 0.375, which is closer to 0.5 than 0.25. As such, it may be more difficult to distinguish somatic mutations from germline mutations based on the VAF at the purity p2 compared to the purity p1 in diploid scenarios.

In the clonal deletion scenario of the second graph 404, at the purity p1, the germline plot 416 and the dashed somatic plot 420 overlap such that there is no separation between the two types of mutations. Thus, it may not be possible to distinguish between germline mutations and somatic mutations based on the VAF at the purity p1 in this scenario. In contrast, there is a relatively large separation between the germline plot 416 and the dashed somatic plot 420 at the purity p2. As such, although the purity p2 is less effective for separating germline mutations and somatic mutations based on the VAF in the first graph 402, the separation between the germline plot 416 and the dashed somatic plot 420 makes the purity p2 more effective for distinguishing between germline mutations and somatic mutations in the second graph 404.

Thus, in accordance with the techniques described herein, a statistical method is used to classify somatic and germline mutations from bulk DNA sequencing data from tumors, including those that lack corresponding germline control samples (referred to herein as “tumor-only” samples). In at least one implementation, the statistical method enables germline versus somatic mutation classification to be determined based on a likelihood ratio using models of germline mutations versus somatic mutations. Moreover, in one or more implementations, joint evidence is used from multiple tumor samples from the same patient in order to increase or recover classification power across the genome.

### Context-Specific Tumor-Only Mutation Classification

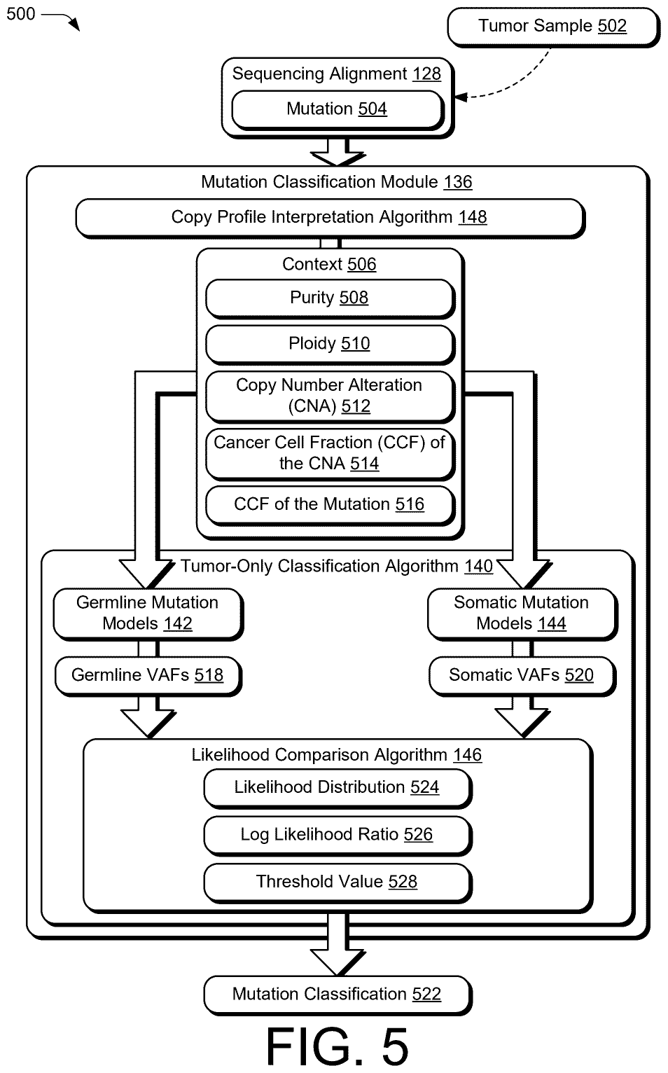

FIG. 5 depicts an example implementation 500 of the tumor-only classification algorithm 140 of the mutation classification module 136 of FIG. 1 in greater detail. The example implementation 500 includes, from FIG. 1, the sequencing alignment 128, the mutation classification module 136, the tumor-only classification algorithm 140, the germline mutation models 142, the somatic mutation models 144, the likelihood comparison algorithm 146, and the copy profile interpretation algorithm 148.

In the example implementation 500, the mutation classification module 136 receives the sequencing alignment 128, or at least a portion thereof, of a sequenced tumor sample 502. The sequencing alignment 128 includes a mutation 504, e.g., as identified by the mutation identification module 130 of FIG. 1. It is to be appreciated that the sequencing alignment 128 may include more than one mutation 504, and mutations at respective locations may be individually evaluated by the mutation classification module 136 to determine their individual classifications. By way of example, the mutation classification module 136 may receive information from the sequencing alignment 128 regarding the mutation 504, its genomic location, the alternate count 208 of the mutation 504, the reference count 210 at the genomic location of the mutation 504, and so forth.

The copy profile interpretation algorithm 148 determines a context 506 of the mutation 504. The context 506 takes into account properties of the tumor sample 502 as well as alterations at the local region of the mutation 504, such as determined based on the sequencing alignment 128. The context 506 includes one or more of each of a purity 508, a ploidy 510, a copy number alteration (CNA) 512, a cancer cell fraction (CCF) of the CNA 514, and a CCF of the mutation 516. As mentioned above with respect to FIGS. 3 and 4, the purity 508 is the percentage or fraction of the cells in the tumor sample 502 that came from tumor cells, as tumor samples often include tumor cells intermixed with an unknown fraction of normal cells. The ploidy 510 refers to the average number of copies of the genome in the tumor sample 502. For instance, normal cells typically have a ploidy of two (diploid) for autosomes (as well as two X chromosomes in females and one X and one Y in males), whereas tumor cells may deviate from two due the amplification or deletion of some parts of the genome. The CNA 512 refers to a change in the number of copies of a specific genetic region of the mutation 504. The CCF of the CNA 514 refers to the fraction of tumor cells in the tumor sample 502 that include the CNA 512, and the CCF of the mutation 516 refers to the fraction of tumor cells that include the mutation 504. Together, the CCF of the CNA 514 and the CCF of the mutation 516 account for heterogeneity in the cancer cell population of the tumor sample 502.

In at least one implementation, the copy profile interpretation algorithm 148 is configured to analyze read-depth information from the sequencing alignment 128 and generate candidate interpretations of a copy profile of the tumor sample 502 that enable the CNA 512 to be inferred in an allele-specific manner. The copy profile interpretation algorithm 148 may be further configured to return candidate solutions for the purity 508 and the ploidy 510 based on the copy profile and select respective values for the purity 508 and the ploidy 510 from the candidate solutions based in part on how well those values fit the raw copy number data. For instance, the best-fitting values may be selected. For a given purity 508 and ploidy 510 solution, the copy profile interpretation algorithm 148 may be further configured to infer the CNA 512, the CCF of the CNA 514, and/or the CCF of the mutation 516.

The context 506 output by the copy profile interpretation algorithm 148 is used by the germline mutation models 142 and the somatic mutation models 144 of the tumor-only classification algorithm 140 to generate germline VAFs 518 and somatic VAFs 520, respectively. For instance, the observed VAFs of germline and somatic mutations are often different with respect to each other and also vary based on how the mutation 504 arises (e.g., heterozygous versus homozygous for germline mutations, whether the mutation occurs before or after a copy number alteration event, and so forth). Accordingly, the different germline mutation models 142 and somatic mutation models 144 model the various ways in which the mutation 504 can arise given the read support of the sequencing alignment 128. The germline VAFs 518 output by the germline mutation models 142 are expected variant allele fraction values according to respective germline models of the mutation 504 having the context 506. Similarly, the somatic VAFs 520 output by the somatic mutation models 144 are expected variant allele fraction values according to respective somatic models of the mutation 504 having the context 506. Example implementations of the germline mutation models 142 and the somatic mutation models 144 will be described in detail below with respect to FIGS. 6A-6C.

The germline VAFs 518 and the somatic VAFs 520 are used by the likelihood comparison algorithm 146 to determine a mutation classification 522 of the mutation 504, which may be output as part of the classified mutations 138 of FIG. 1, for example. The mutation classification 522 indicates whether the mutation 504 is a germline mutation or a somatic mutation. To determine the mutation classification 522, in at least one implementation, the likelihood comparison algorithm 146 calculates a likelihood that a given germline or somatic model fits the observed data of the sequencing alignment 128 by modeling a likelihood distribution 524, which includes a distribution of the observed data; calculating a log likelihood ratio 526 to determine whether a germline or somatic model better fits the observed data; and classifying the mutation 504 based on a threshold value 528.

In one or more implementations, the likelihood comparison algorithm 146 utilizes a beta binomial distribution to model the distribution of the observed data of the sequencing alignment 128. For instance, consider that the observed VAF of the sequencing alignment 128, v_{obs}, can be calculated as:

$${v}_{o b s} = \frac{{n}_{a l t}}{{n}_{a l t} + {n}_{ref}}$$

where nₐₗₜ is a first number of reads that support the mutation 504 (e.g., the alternate count 208 of FIG. 2) and n_{ref} is a second number of reads that support the reference allele (e.g., the reference count 210 of FIG. 2). However, v_{obs} may not be an accurate estimation of the true VAF of the mutation, v*, because sequencing is a random sampling process that can result in unequal numbers of reads being generated for different molecules of DNA extracted from the tumor sample 502. There is a true discrete number of reads that support the mutation 504 in the tumor sample 502,

$${n}_{a l t}^{*} ,$$

because there is a discrete number of cells and units of the genome in the tumor sample 502. Therefore, the beta binomial distribution models this value as:

$${n}_{a l t}^{*} ∼ B e t a B i n o m ( N = {n}_{a l t} + {n}_{r e f} , a = {n}_{a l t} + 1 , b = {n}_{r e f} + 1 )$$

where N is the read coverage, a is a first shape parameter of the beta distribution that represents the reads that support the mutation 504, and b is a second shape parameter of the beta distribution that represents the reference allele. The resulting distribution described herein, V, is a statistical model that describes the distribution of the true measurement of the mutation 504

$$( e . g . , {n}_{a l t}^{*} or {v}^{*} ) .$$

In at least one variation, however, another type of likelihood distribution is used. As such, the beta binomial distribution is one example used by the likelihood comparison algorithm 146 to model the distribution of the true measurement of the mutation 504 according to the techniques described herein.

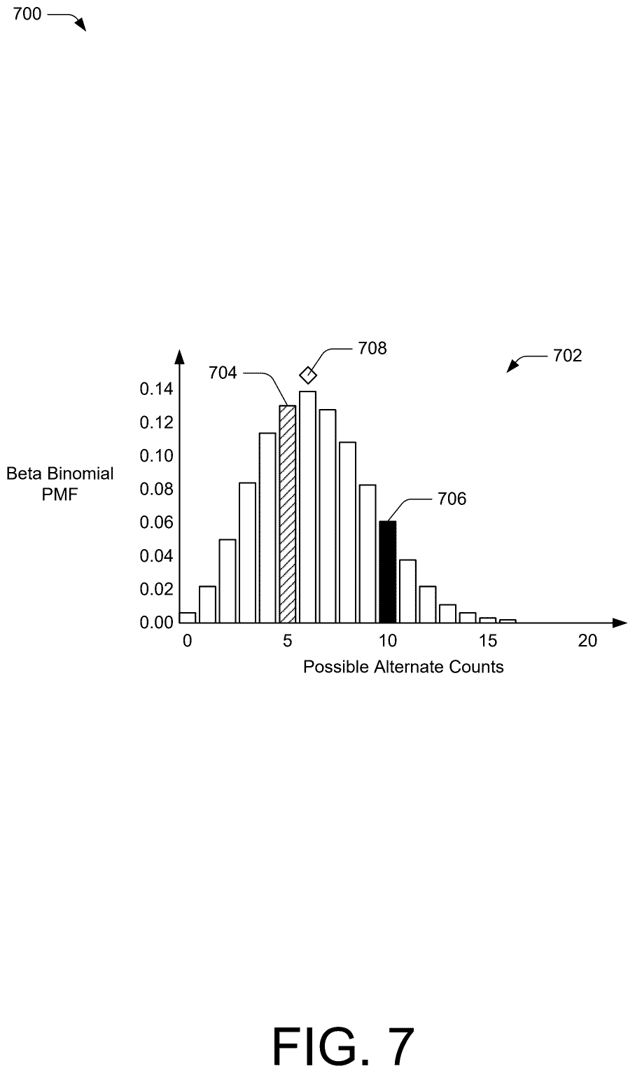

In at least one implementation, once the distribution of the true measurement (V) is determined, the likelihood comparison algorithm 146 assesses how well a given model (M) fits the distribution V. By way of example, the likelihood comparison algorithm 146 may calculate the likelihood of model M given the observed data of the sequencing alignment 128 and based on the probability density function (PDF) or probability mass function (PMF) of V. For instance, the VAF of a given model (v_{M}) may be calculated as a function of the context 506 according to:

$${v}_{M} = {f}_{M} ( C )$$

where C is the context 506. Given this, the likelihood of observing the data under the model M may be expressed as:

$$L ( {v}_{M} ; {n}_{alt} , {n}_{ref} , C ) = {P}_{V ( {n}_{alt} , {n}_{ref} )} ( {f}_{M} ( C ) )$$

where L(v_{M}; nₐₗₜ, n_{ref}, C) represents a likelihood function for the model M explaining the observed data (nₐₗₜ, n_{ref}, C) for the calculated v_{M}, and P_{V(n}_{ₐₗₜ}_{,n}_{_{ref}}₎(f_{M}(C)) represents the probability of observing the v_{M} given the distribution V. The likelihood may be mapped to the distribution V to generate the likelihood distribution 524, an example of which will be described with respect to FIG. 7. For example, the model M may be mapped to a number of alternate counts on the distribution V based on the calculated v_{M} and the read total coverage (e.g., nₐₗₜ+n_{ref}).

The likelihood comparison algorithm 146 may calculate the corresponding likelihood for each of the germline mutation models 142 and the somatic mutation models 144 and select a best (e.g., highest likelihood) germline mutation model (M_{germ}) and a best somatic mutation model (Mₛₒₘ) out of the possible germline mutation models 142 and somatic mutation models 144, respectively. By way of example, the likelihood comparison algorithm 146 may use the following equations to select M_{germ} and Mₛₒₘ:

$$\begin{matrix}{M}_{g e r m} = arg {max}_{g ∈ G} d ( {v}_{g} ; {n}_{a l t} , {n}_{r e f} , C ) \\ {M}_{s o m} = arg {max}_{s ∈ S} d ( {v}_{s} ; {n}_{a l t} , {n}_{r e f} , C )\end{matrix}$$

where d is a likelihood function and argmax denotes the argument (the value of the variable g or s) at which the likelihood function achieves its maximum. That is, for each possible germline mutation model g in the set of germline mutation models G (e.g., the germline mutation models 142), and for each somatic mutation model s in the set of somatic mutation models S (e.g., the somatic mutation models 144), the likelihoods are calculated based on the observed data (e.g., the counts of the alternate alleles nₐₗₜ and the reference alleles n_{ref}). The likelihood d(v_{g}; nₐₗₜ, n_{ref}, C) may be computed for each model g of the germline mutation models 142 (e.g., term G), where v_{g} is the VAF of the mutation 504 according to the model g. Similarly, the likelihood d(vₛ; nₐₗₜ, n_{ref}, C) may be computed for each model s of the somatic mutation models 144 (e.g., term S), where vₛ is the VAF of the mutation 504 according to the model s.

The germline model that maximizes the likelihood is selected as the best germline mutation model M_{germ} from among the germline mutation models 142. In other words, M_{germ} is chosen as the germline mutation model with the highest likelihood given the observed data of the sequencing alignment 128. Similarly, the somatic model that maximizes the likelihood is selected as the best somatic mutation model Mₛₒₘ from among the somatic mutation models 144. That is, the somatic mutation model with the highest likelihood given the observed data of the sequencing alignment 128 is selected as Mₛₒₘ.

Once M_{germ} and Mₛₒₘ are selected, the likelihood comparison algorithm 146 may compute the log likelihood ratio 526 based on a ratio of their likelihoods as:

$$L R ( {M}_{g erm} , {M}_{s o m} ; {n}_{alt} , {n}_{ref} , C ) = \frac{L ( {v}_{{M}_{g e r m}} ; {n}_{alt} , {n}_{ref} , C )}{L ( {v}_{{M}_{s o m}} ; {n}_{a l t} , {n}_{r e f} , C )}$$

$$log LR ( {M}_{g e r m} , {M}_{s o m} ; {n}_{a l t} , {n}_{r e f} , C ) = log L ( {v}_{{M}_{g e r m}} ; {n}_{a l t} , {n}_{r e f} , C ) - log L ( {v}_{{M}_{s o m}} ; {n}_{a l t} , {n}_{r e f} , C )$$

where LR(M_{germ}, Mₛₒₘ; nₐₗₜ, n_{ref}, C) is the likelihood ratio (e.g., the likelihood of the germline mutation model M_{germ} divided by the likelihood of the somatic mutation model Mₛₒₘ) and log LR(M_{germ}, Mₛₒₘ; nₐₗₜ, n_{ref}, C) is the log likelihood ratio 526. Taking the logarithm of the likelihood ratio aids in interpretation, as a log likelihood ratio (e.g., log odds ratio) of zero indicates that both models equally fit the data. A negative value for the log likelihood ratio 526 indicates that the somatic mutation model Mₛₒₘ better fits the data, and thus the mutation 504 is more likely to be a somatic mutation. Conversely, a positive value for the log likelihood ratio 526 indicates that the germline mutation model M_{germ} better fits the data, and thus the mutation 504 is more likely to be a germline mutation. Thus, the log likelihood ratio 526 indicates a relative fit of the germline mutation model and the somatic mutation model to the alternate count 208 and reference count 210 data given the context 506.

In at least one implementation, the likelihood comparison algorithm 146 further sets and uses the threshold value 528 to determine the mutation classification 522. As a non-limiting example, the threshold value 528 is set to zero. However, in some instances, setting the threshold value 528 to zero may lead to low sensitivity for true somatic mutations, such as when M_{germ} and Mₛₒₘ have similar or near-equal VAFs. For example, M_{germ} and Mₛₒₘ may have similar or near-equal VAFs when the tumor sample has high purity, and so clonal somatic mutations (e.g., occurring in substantially all tumor cells) and germline mutations would occur in the same number of cells and thus be difficult to distinguish from one another. Accordingly, in at least one implementation, the likelihood comparison algorithm 146 calculates the threshold value 528 per mutation in order to utilize the context 506.

As mentioned above, each model has a corresponding VAF calculated as v_{M}=f_{M}(C), and the expected number of alternate reads

$${\hat{n}}_{a l t}^{( M )}$$

supporting a mutation arising from model M follows a binomial distribution according to:

$$\begin{matrix}{\hat{n}}_{a l t}^{( M )} ∼ B i n o m ( n , {f}_{M} ( C ) ) \\ {\hat{n}}_{r e f}^{( M )} = n - {\hat{n}}_{a l t}^{( M )}\end{matrix}$$

where n is the sequencing depth. For each possible

$${\hat{n}}_{a l t}^{( M )} ,$$

the likelihood comparison algorithm 146 may further calculate an expected log likelihood as:

$$log LR ( {M}_{g e r m} , {M}_{s o m} ; {\hat{n}}_{a l t}^{( M )} , {\hat{n}}_{r e f}^{( M )} , C ) = log L ( {v}_{{M}_{g e r m}} ; {\hat{n}}_{a l t}^{( M )} , {\hat{n}}_{r e f}^{( M )} , C ) - log L ( {v}_{{M}_{s o m}} ; {\hat{n}}_{a l t}^{( M )} , {\hat{n}}_{r e f}^{( M )} , C )$$

to generate a distribution Y_{M} having a linearly transformed x-axis

$$( e . g . , {Y}_{M} = log LR ( {M}_{g e r m} , {M}_{s o m} ; {\hat{n}}_{a l t}^{( M )} , {\hat{n}}_{r e f}^{( M )} , C ) ) ,$$

which represents the expected distribution of log likelihood ratio values for a mutation derived from the corresponding model and detected with sequencing. This log likelihood ratio distribution may be computed for M_{germ} and/or Mₛₒₘ, resulting in Y_{germ} and/or Yₛₒₘ, respectively.

The threshold value 528, T, may be calculated based on a desired performance metric and using one or both of the distributions Y_{germ} and Yₛₒₘ. For example, the threshold value 528 may be calculated for a desired or acceptable sensitivity for classifying somatic mutations as somatic (e.g., a sensitivity in a range from 90% to 99%). Sensitivity may also be referred to as a true positive rate. As a non-limiting example, the minimum threshold value 528 may be calculated such that:

$$Sensitivity = {\sum }_{l = - ∞}^{T} {Y}_{s o m} ( l )$$

where l is a log likelihood ratio (LR) value and Yₛₒₘ is the LR probability distribution for the somatic model. In practice, T may be calculated such that:

$$T = arg {min}_{t} ( ( {\sum }_{l = - ∞}^{t} {Y}_{s o m} ( l ) ) - Sensitivity \geq 0 )$$

where t is a possible threshold value, and argminₜ finds the minimum value of t so that the cumulative sum of LR values up to t is at least the desired sensitivity. Given T, an expected false positive rate (FPR) may be calculated as:

$$F P R = {\sum }_{l = - ∞}^{T} {Y}_{g e r m} ( l )$$

where the FPR corresponds to the number or percentage of germline mutations that are inaccurately classified as somatic mutations.

Additionally, or alternatively, the threshold value 528 may be set to a desired or acceptable false positive rate for classifying germline mutations as somatic, and the expected sensitivity for somatic mutations may then be calculated. For example, the following equations may be used:

$$\begin{matrix}FPR = {\sum }_{l = - ∞}^{T} {Y}_{g e r m} ( l ) \\ T = arg {min}_{t} ( ( {\sum }_{l = - ∞}^{T} {Y}_{g e r m} ( l ) ) - FPR \geq 0 ) \\ Sensitivity = {\sum }_{l = - ∞}^{T} {Y}_{s o m} ( l )\end{matrix}$$

where argminₜ finds the threshold value 528 that minimizes value of t so that the cumulative sum of LR up to t for Y_{germ} is greater than the desired FPR, thus optimizing the threshold in terms of germline mutations rather than somatic mutations.

The likelihood comparison algorithm 146 may compare the log likelihood ratio 526 to the threshold value 528 to determine the mutation classification 522 of the mutation 504. By way of example, the mutation classification 522 may classify the mutation 504 as a germline mutation in response to the log likelihood ratio 526 being greater than or equal to the threshold value 528 or classify the mutation 504 as a somatic mutation in response to the log likelihood ratio 526 being less than the threshold value 528.

In at least one implementation, sequencing data 118 from multiple samples from the same patient may be combined. In such a scenario, the log likelihood ratio 526 from multiple samples may be summed to determine a joint log likelihood ratio of observing the data from both the germline mutation models 142 and the somatic mutation models 144. For example, the joint log likelihood ratio may be calculated according to:

$${N}_{a l t} = {n}_{a l t}^{( 1 )} , … , {n}_{a l t}^{( k )}$$

$${N}_{r e f} = {n}_{r e f}^{( 1 )} , … , {n}_{r e f}^{( k )}$$

$$ℂ = {C}^{( 1 )} , … , {C}^{( k )}$$

$$log {LR}_{j o i n t} ( {M}_{g e r m} , {M}_{s o m} ; {N}_{a l t} , {N}_{r e f} , ℂ ) = {\sum }_{i = 1}^{k} log LR ( {M}_{g e r m} , {M}_{s o m} ; {n}_{a l t}^{( i )} , {n}_{r e f}^{( i )} , {C}^{( i )} )$$

$${M}_{g e r m} = arg {max}_{g ∈ G} {\prod }_{i = 1}^{k} d ( {v}_{g}^{( i )} ; {n}_{alt}^{( i )} , {n}_{ref}^{( i )} , {C}^{( i )} )$$

$${M}_{s o m} = arg {max}_{s ∈ S} {\prod }_{i = 1}^{k} d ( {v}_{s}^{( i )} ; {n}_{alt}^{( i )} , {n}_{ref}^{( i )} , {C}^{( i )} )$$

where there are k samples, and

$$log LR ( {M}_{g erm} , {M}_{s o m} ; {n}_{alt}^{( i )} , {n}_{ref}^{( i )} , {C}^{( i )} )$$

represents the log likelihood ratio of observing the data from the i^{th} sample under the germline model with the highest joint likelihood M_{germ} and the somatic model with the highest joint likelihood Mₛₒₘ. For example, the log likelihood ratio 526 may be calculated separately for samples 1 through k by comparing the same germline model and somatic model, and the joint log likelihood ratio may be the sum of the individual log likelihoods ratios.

To set the threshold value 528 in this scenario, the likelihood comparison algorithm 146 may calculate the sum of the generated LR distributions for the highest joint likelihood germline model and the highest joint likelihood somatic model across the k samples, such as according to:

$$\begin{matrix}J {Y}_{g e r m} = {\sum }_{i}^{k} {Y}_{g e r m}^{( i )} \\ {JY}_{s o m} = {\sum }_{i}^{k} {Y}_{s o m}^{( i )}\end{matrix}$$

where JY_{germ} represents the aggregated or joint likelihood ratio probability distribution for the germline mutation models 142 across k tumor samples, and JYₛₒₘ represents the aggregated or joint likelihood ratio probability distribution for the somatic mutation models 144 across k instances.

The sum of random variables is a convolution, which can be directly calculated. When there are at least three tumor samples (e.g., k>3), JY can be approximated with a normal distribution as:

$$JY ∼ Normal ( μ = {\sum }_{i}^{k} 𝔼 [ {Y}^{( i )} ] , {σ}^{2} = {\sum }_{i}^{k} Var [ {Y}^{( i )} ] )$$

where [symbol] [Y⁽ⁱ⁾] corresponds to the expected LR values across the k samples, μ is the mean, and σ² denotes the variance of the normal distribution.

The threshold value 528 may then be computed as a joint threshold value as described above using JY_{germ} and/or JYₛₒₘ rather than Y_{germ} and/or Yₛₒₘ (e.g., the single tumor sample likelihood ratio distributions), and the mutation classification 522 may be output based on the joint log likelihood ratio relative to the joint threshold value.

FIGS. 6A-6C depict an overview 600 of example germline and somatic mutation models that may be used by the mutation classification module 136 to classify a mutation found in a sequenced tumor sample. The overview 600 includes a first germline mutation model 602, a second germline mutation model 604, and a third germline mutation model 606 depicted in FIG. 6A; a first somatic mutation model 608, a second somatic mutation model 610, and a third somatic mutation model 612 depicted in FIG. 6B; and a fourth somatic mutation model 614, a fifth somatic mutation model 616, and a sixth somatic mutation model 618 depicted in FIG. 6C.

It is assumed that all cells in a tumor sample (e.g., the tumor sample 502 of FIG. 5) originate from the same individual. Therefore, both tumor and normal cells contribute to the total number of sequencing reads that either support a mutation (e.g., the alternate count 208 of FIG. 2) or the reference allele (e.g., the reference count 210 of FIG. 2). As mentioned previously, germline mutations are either heterozygous (e.g., inherited from one parent) or homozygous (e.g., inherited from both parents). Therefore, the expected VAFs (e.g., the germline VAFs 518) are modeled for both heterozygous and homozygous mutations.

In the overview 600, the respective mutation models will be discussed with reference to normal tissue 620, a clonal tumor 622, and a subclone 624. The normal tissue 620 is non-cancerous tissue comprising normal, non-cancerous cells. The clonal tumor 622 refers to a portion of the tumor that originates from an initial tumor cell and is genetically indistinguishable from the initial tumor cell. The subclone 624 refers to a portion of the tumor that has acquired additional mutations or alterations. Cells of the subclone 624 are genetically different than the clonal tumor 622 and genetically different than other subclones. Reference will also be made to a first homolog 626 (e.g., inherited from one parent), a second homolog 628 (e.g., inherited from the other parent), and a mutation 630. The mutation 630 may occur on one or both of the first homolog 626 and the second homolog 628, e.g., in the same genetic locus.

Before discussing the differences between the respective mutation models in detail, it is to be appreciated that the models may estimate an expected amount of DNA of the region where the mutation 630 resides. For example, the expected amount of DNA, D, may be estimated as:

$$D = α \cdot ( ω \cdot ( NA + N B ) + ( 1 - ω ) \cdot 2 ) + ( 1 - α ) \cdot 2 + e$$

where NA refers to the homolog with the smaller number of copies on average in the whole sample (e.g., the first homolog 626, which is the minor allele in this scenario), NB refers to the homolog with the larger number of copies on average in the whole sample (e.g., the second homolog 628, which is the major allele in this scenario), α is the purity 508, ω is the CCF of the CNA 514, t is the ploidy 510, and e estimates the relative amount of DNA contamination. For instance, the above equations consider that a portion of the cells in the tumor sample 502 may be tumor cells (e.g., a, or the purity 508), and so the calculation of D weights the total amount of DNA contributed by the tumor. The remaining cells (1−α) are normal cells (e.g., the normal tissue 620). The term NA+NB refers to the total amount of DNA in the subset of the tumor cells with a copy number alteration (CNA) event (ω, or the CCF of the CNA 514). The remaining tumor cells without the CNA event (1−ω) are expected to have two copies of the allele. The relative amount of DNA contamination e may be calculated as:

$$e = \frac{q \cdot ( τ \cdot α + ( 1 - α ) \cdot 2 )}{1 - q}$$

where q is an estimated contamination rate.

D can also be expressed such that each homolog has its own mixture between two states:

$$D = α \cdot ( {ω}_{N A} \cdot {NA}^{′} + {ω}_{N B} \cdot {NB}^{′} + ( 1 - {ω}_{N A} ) \cdot {NA}^{″} + ( 1 - {ω}_{N B} ) \cdot {NB}^{″} ) + ( 1 - α ) \cdot 2 + e$$

where ω_{NA} and ω_{NB} correspond to the CCF of NA and NB, respectively, NA′ and NA″ correspond to the two integer states for the minor homolog, and NB′ and NB″ correspond to the two integer states of the major homolog. Note that when ω_{NA}=ω_{NB} and NA″≈NB″≈1, the equation is equivalent to the previous equation for D.

As discussed previously herein, germline mutations are inherited and are present in substantially all cells of the body. Referring first to FIG. 6A, the first germline mutation model 602 (e.g., of the germline mutation models 142) shows a heterozygous germline mutation. That is, the mutation 630 occurs on one homolog, e.g., the second homolog 628. Because the mutation 630 is present in the normal tissue 620, the clonal tumor 622 also includes the mutation 630 on the second homolog 628. The subclone 624 has undergone a CNA event such that the first homolog 626 is replicated, resulting in a first copy of the first homolog 626(1) and a second copy of the first homolog 626(2).

The second germline mutation model 604 also shows a heterozygous germline mutation, with the mutation 630 occurring on the second homolog 628. However, in the second germline mutation model 604, the second homolog 628 has undergone the copy number alteration in the subclone 624, resulting in a first copy of the second homolog 628(1) and a second copy of the second homolog 628(2), which both include the mutation 630.

In order to account for the mutation 630 being on either of the two possible homologs and potential copy number alteration events thereof, the first germline mutation model 602 and the second germline mutation model 604 may calculate the VAF using the following equations:

$$\begin{matrix}{f}_{{G}_{N A}} = \frac{α \cdot [ ω \cdot NA + ( 1 - ω ) ] + ( 1 - α )}{D} \\ {f}_{{G}_{N B}} = \frac{α \cdot [ ω \cdot NB + ( 1 - ω ) ] + ( 1 - α )}{D}\end{matrix}$$

where NA refers to the first homolog 626, NB refers to the second homolog 628, α is the purity 508, ω is the CCF of the CNA 514, and D is the expected amount of DNA, as described above. By way of example, the first germline mutation model 602 may calculate the VAF for the first homolog 626 (e.g., f_{G}_{_{NA}}), and the second germline mutation model 604 may calculate the VAF for the second homolog 628 (f_{G}_{_{NB}}), or vice versa. Note that in both models, the scale factor for both (1−α) and (1−ω) is 1 because normal cells and tumor cells without a CNA event contribute a single copy of a germline heterozygous mutation.

Thus, together, the first germline mutation model 602 and the second germline mutation model 604 account for whether the mutation 630 is present on the homolog that undergoes a CNA event or the homolog that does not undergo the CNA event.

The third germline mutation model 606 (e.g., of the germline mutation models 142) depicts a homozygous germline mutation. That is, the mutation 630 is present on both the first homolog 626 and the second homolog 628 in the normal tissue 620 as well as the clonal tumor 622. The subclone 624 has undergone a CNA event of the first homolog 626, resulting in the first copy of the first homolog 626(1) and the second copy of the first homolog 626(2). However, unlike the first germline mutation model 602, because the mutation 630 also present on the first homolog 626, the mutation 630 undergoes a CNA and is present in three copies in the subclone 624.

Using the third germline mutation model 606, the VAF may be calculated as:

$${f}_{{G}_{hom}} = \frac{α \cdot [ ω \cdot ( NA + NB ) + ( 1 - ω ) \cdot 2 ] + ( 1 - α ) \cdot 2}{D}$$

where the scale factor for both (1−α) and (1−ω) is 2 because normal cells and tumor cells without a CNA event contribute two copies of a germline homozygous mutation. Moreover, f_{G}_{ₙₒₘ} is approximately equal to one since the numerator approximates D.

Unlike the germline mutations, somatic mutations are modeled to exist in the tumor cells (and not the normal cells) of the tumor sample 502. Additionally, CNA events are assumed to occur in the tumor cells, and not the normal cells. The order of these events (e.g., a mutation before a CNA event, or the CNA event before the mutation) may affect the VAF of the mutation, which is accounted for in the somatic mutation models 144.

Referring now to FIG. 6B, the first somatic mutation model 608 depicts a first subclone 624(1) and a second subclone 624(2). For instance, as the clonal tumor 622 divides, individual cells may undergo independent mutations or CNA events. The mutation 630 occurs in the first subclone 624(1) (e.g., on the second homolog 628) and not in the second subclone 624(2). A CNA event occurs in the second subclone 624(2), resulting in duplication of the second homolog 628 represented as the first copy of the second homolog 628(1) and the second copy of the second homolog 628(2). Neither of the first copy of the second homolog 628(1) and the second copy of the second homolog 628(2) includes the mutation 630 because the mutation 630 has occurred separately in the first subclone 624(1).

The first somatic mutation model 608 (e.g., of the somatic mutation models 144) estimates the VAF for the mutation 630 occurring in the tumor cells without a CNA event. By way of example, the first somatic mutation model 608 may estimate the VAF according to:

$${f}_{{S}_{i n d}} = \frac{α \cdot ( 1 - ω ) \cdot μ}{D} , μ \leq ( 1 - ω )$$

where α is the purity 508, ω is the CCF of the CNA 514, μ is the CCF of the mutation 516, and D is the expected amount of DNA, as described above. Unlike the germline mutation models 142, the somatic mutation models 144, including the first somatic mutation model 608, do not include the (1−α) term in the numerator because the normal tissue 620 does not include the mutation 630. Instead, the first somatic mutation model 608, and the rest of the somatic mutation models 144, utilize the CCF of the mutation 516 (μ), as this is variable for individual somatic mutations.

The second somatic mutation model 610 calculates the VAF for the mutation 630 occurring after the CNA event. In the second somatic mutation model 610 depicted in the example overview 600, the second subclone 624(2) is a mutated subclone of the first subclone 624(1) that has undergone duplication of the second homolog 628. In the depicted example, the mutation 630 is located on the second copy of the second homolog 628(2). However, it is to be appreciated that whether the mutation 630 is on first homolog 626, the first copy of the second homolog 628(1), or the second copy of the second homolog 628(2) does not affect the calculation of the VAF since each homolog contributes one copy of the mutation 630 in this scenario. By way of example, the second somatic mutation model 610 may calculate the VAF according to:

$${f}_{{S}_{CNA \to mut}} = \frac{α \cdot μ}{D} , μ \leq ω$$

where α is the purity 508, μ is the CCF of the mutation 516, and D is the expected amount of DNA, as described above. Note that ω, the CCF of the copy number alteration, is included in the calculation of D.

The third somatic mutation model 612 (FIG. 6B) and the fourth somatic mutation model 614 (FIG. 6C) account for mutations and CNA events that happen around the same time (e.g., in the same subclone). For example, the third somatic mutation model 612 and the fourth somatic mutation model 614 both depict the clonal tumor 622 as having a single copy of the first homolog 626 and the second homolog 628 and no mutations present. In the third somatic mutation model 612, the second homolog 628 is duplicated in the subclone 624, resulting in the first copy of the second homolog 628(1) and the second copy of the second homolog 628(2), which both include the mutation 630. In contrast to this, in the fourth somatic mutation model 614, the first homolog 626 is duplicated in the subclone 624, resulting in the first copy of the first homolog 626(1) and the second copy of the first homolog 626(2). Like in the third somatic mutation model 612, the mutation 630 is present on the second homolog 628 in the subclone 624; however, there is a single copy of the second homolog 628.

In order to account for the mutation 630 being on either of the two possible homologs and potential copy number alteration events thereof, the third somatic mutation model 612 and the fourth somatic mutation model 614 may calculate the VAF using the following equations:

$$\begin{matrix}{f}_{{S}_{N A + m u t}} = \frac{α \cdot μ \cdot NA}{D} , μ \leq ω \\ {f}_{{S}_{N B + m u t}} = \frac{α \cdot μ \cdot NB}{D} , μ \leq ω\end{matrix}$$

By way of example, the third somatic mutation model 612 may be used to calculate the VAF for the first homolog 626 (e.g., NA, the homolog that is not amplified), and the fourth somatic mutation model 614 may be used to calculate the VAF for the second homolog 628(e.g., NB, the homolog that is amplified). In the third somatic mutation model 612 and the fourth somatic mutation model 614, the number of copies of the mutation 630 is weighted by the resulting number of copies of the homolog on which it resides after the CNA event. Note that in these scenarios, μ=ω, so only one term is used.

If the mutation 630 occurs prior to a CNA event, the CNA may occur in a subset of cells having the mutation 630. For example, the fifth somatic mutation model 616 and the sixth somatic mutation model 618 show the first subclone 624(1) having the mutation 630 on second homolog 628. In the fifth somatic mutation model 616, the second homolog 628 then undergoes a CNA event in the second subclone 624(2) such that both of the first copy of the second homolog 628(1) and the second copy of the second homolog 628(2) include the mutation 630. In contrast, the sixth somatic mutation model 618 shows the first homolog 626 undergoing the CNA event in the second subclone 624(2).

In order to account for these differences, the fifth somatic mutation model 616 and the sixth somatic mutation model 618 may calculate the VAF using the following equations:

$$\begin{matrix}{f}_{{S}_{m u t \to N A}} = \frac{α \cdot μ \cdot ( {ω}_{μ} \cdot NA + ( 1 - {ω}_{μ} ) )}{D} , μ \geq min ( ω , 1 - ω ) \\ {f}_{{S}_{m u t \to N B}} = \frac{α \cdot μ \cdot ( {ω}_{μ} \cdot NB + ( 1 - {ω}_{μ} ) )}{D} , μ \geq min ( ω , 1 - ω ) \\ {ω}_{μ} = \frac{ω}{μ}\end{matrix}$$

where ω_{μ} is the fraction of tumor cells that have the mutation with the CNA event, where it is assumed that ω≤μ. By way of example, the fifth somatic mutation model 616 may be used to calculate the VAF for the first homolog 626 (e.g., NA), and the sixth somatic mutation model 618 may be used to calculate the VAF for the second homolog 628 (e.g., NB). Similar to the third somatic mutation model 612 and the fourth somatic mutation model 614, the CNA event is weighted by the resulting number of copies of the homolog (e.g., NA or NB). Remaining tumor cells with the mutation but not the CNA event have one copy of the mutation 630.

It is to be appreciated that the example locations of the mutation 630 depicted in the overview 600 are illustrative in order to demonstrate the way in which different types of mutations can arise, and variations are possible without departing from the spirit or scope of the described techniques.

Having discussed example details of the techniques for context-specific tumor-only mutation classification, consider now examples to illustrate usage of the techniques.

### Example Applications

FIG. 7 depicts an illustrative example 700 of calculating likelihoods of models given observed tumor sequencing data. The illustrative example 700 includes a likelihood distribution graph 702, which relates possible alternate counts (horizontal axis, also referred to as n*ₐₗₜ herein) to a beta binomial PMF (vertical axis). For example, the likelihood distribution graph 702 depicts a model of the distribution of n*ₐₗₜ based on the observed data (e.g., of the sequencing alignment 128), such as described above with respect to FIG. 5. The likelihood distribution graph 702 represents one example implementation of the likelihood distribution 524. The likelihood distribution graph 702 includes a somatic model likelihood 704 (e.g., striped bar) and a germline model likelihood 706 (e.g., black-filled bar) mapped to the distribution of n*ₐₗₜ. The somatic model likelihood 704 corresponds to the beta binomial PMF for the highest likelihood somatic mutation model Mₛₒₘ based on its calculated VAF (e.g., v_{M}_{ₛₒₘ}), and the germline model likelihood 706 corresponds to the beta binomial PMF for the highest likelihood germline mutation model M_{germ} based on its calculated VAF (e.g., v_{M}_{_{germ}}). The likelihood distribution graph 702 further includes a symbol 708 indicating an observed alternate count. In the example depicted in FIG. 7, the observed alternate count is six, and the reference count is fourteen for a total coverage of twenty reads.

In the non-limiting, illustrative example, the VAF of the highest likelihood somatic mutation model (e.g., v_{M}_{ₛₒₘ}) is 0.25, and so the somatic model likelihood 704 is mapped to five alternate counts based on the total coverage (e.g., five expected alternate counts divided by the total coverage of twenty reads is 0.25). Continuing with this example, the VAF of the highest likelihood germline model (e.g., v_{M}_{_{germ}}) is 0.5, and so the germline model likelihood 706 is mapped to ten alternate counts (e.g., ten expected alternate counts divided by the total coverage of twenty reads is 0.5). Thus, the somatic model likelihood 704 and the germline model likelihood 706 are mapped to the corresponding number of alternate counts based on the expected VAFs of the mutation for the respective models and the total coverage.

The somatic model likelihood 704 is 0.13 in the present non-limiting example, and the germline model likelihood 706 is 0.06. This results in a log likelihood ratio value (e.g., the log likelihood ratio 526) of −0.77 (e.g., less than zero), which indicates that the distribution better fits the highest likelihood somatic mutation model Mₛₒₘ than the highest likelihood germline mutation model M_{germ}. Thus, in the illustrative example 700, the mutation classification 522 may be somatic. In some instances, however, it may be desirable to calculate a threshold (e.g., the threshold value 528 of FIG. 5) that is non-zero in order to classify the mutation more accurately and control classification performance.

It is to be appreciated that although the likelihood distribution graph 702 is depicted as a bar graph, the likelihood distribution graph 702 may be visualized in other ways, such as a line graph. Moreover, the likelihood distribution 524 and the log likelihood ratio 526 may be determined without explicitly visualizing the values in graphic form.

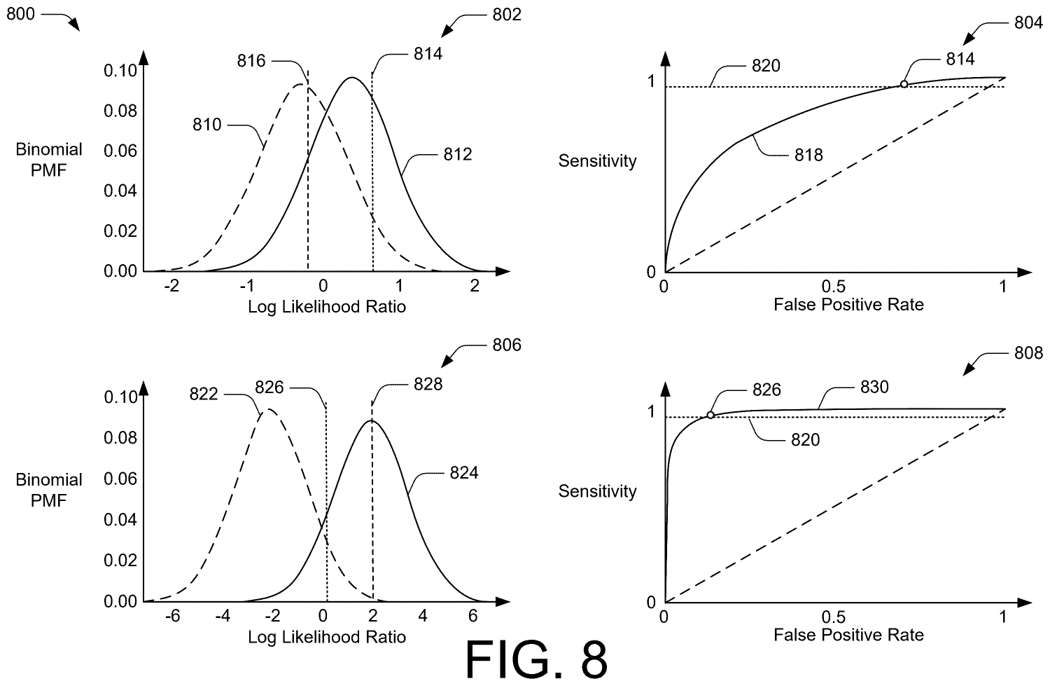

FIG. 8 depicts illustrative examples 800 of using simulated germline and somatic log likelihood distributions to determine a threshold for classifying a mutation observed in tumor sequencing data. The illustrative examples 800 include a first simulated log likelihood ratio graph 802, a first theoretical performance graph 804, a second simulated log likelihood ratio graph 806, and a second theoretical performance graph 808. The first theoretical performance graph 804 is a theoretical performance graph for the first simulated log likelihood ratio graph 802, and the second theoretical performance graph 808 is a theoretical performance graph for the second simulated log likelihood ratio graph 806. The first simulated log likelihood ratio graph 802 is generated for a first mutation having a first context, and the second simulated log likelihood ratio graph 806 is generated for a second mutation having a second context that is different than the first context. As such, the first simulated log likelihood ratio graph 802 and the second simulated log likelihood ratio graph 806 are independent from one another.

The first simulated log likelihood ratio graph 802 relates a log likelihood ratio (horizontal axis, also referred to as a log odds ratio) to a beta binomial PMF (vertical axis) and includes a first simulated somatic mutation model LR distribution 810 and a first simulated germline mutation model LR distribution 812. The first simulated somatic mutation model LR distribution 810 corresponds to a highest likelihood somatic mutation model Mₛₒₘ for the first mutation based on the first context, and the first simulated germline mutation model LR distribution 812 corresponds to a highest likelihood germline mutation model M_{germ} for the first mutation based on the first context. For example, the first simulated somatic mutation model LR distribution 810 and the first simulated germline mutation model LR distribution 812 represent the terms Yₛₒₘ and Y_{germ} (e.g., as described with respect to FIG. 5), respectively, for the first mutation. The first simulated log likelihood ratio graph 802 further includes a first threshold 814 for classifying the first mutation and a first observed log likelihood ratio 816 (e.g., the log likelihood ratio 526 determined for the first mutation).

The first simulated somatic mutation model LR distribution 810 and the first simulated germline mutation model LR distribution 812 have substantial overlap, indicating the highest likelihood somatic mutation model Mₛₒₘ and the highest likelihood germline mutation model M_{germ} for the first mutation will likely produce a very similar log likelihood ratio value. This is also indicated in the first theoretical performance graph 804, which relates a false positive rate (horizontal axis, corresponding to putative germline mutations that are classified as somatic) to sensitivity (vertical axis, corresponding to putative somatic mutations that are classified as somatic). The first theoretical performance graph 804 includes a first curve 818 and a desired sensitivity 820 (e.g., 95% sensitivity). The first curve 818 is a receiver operating characteristic (ROC) curve, where each point on the first curve 818 represents a different potential threshold value setting (e.g., the term t described with respect to FIG. 5) for classifying the first mutation. For example, as the threshold value for the classification changes, the sensitivity and false positive rate values vary. The diagonal line from the bottom left to the top right represents random guessing.

In the present example, the first threshold 814 is set based on the desired sensitivity 820, e.g., so that the sensitivity of classifying the first mutation is not less than the desired sensitivity 820. The first threshold 814 is depicted as a point on the first curve 818, indicating that the false positive rate is relatively high at this threshold value. This is also reflected in the first simulated log likelihood ratio graph 802, as a relatively large portion of the first simulated germline mutation model LR distribution 812 is less than the first threshold 814. As such, in order to classify a somatic mutation with high (e.g., greater than 95%) sensitivity, a germline mutation is also relatively likely to be classified as a somatic mutation (e.g., the FPR is greater than 50%).

In the example depicted in the first simulated log likelihood ratio graph 802, the first threshold 814 has a value of 0.65, and the first observed log likelihood ratio 816 has a value of −0.19. Using these example values, the first mutation may be classified as somatic because the first observed log likelihood ratio 816 is less than the first threshold 814.

The second simulated log likelihood ratio graph 806 relates a log likelihood ratio (horizontal axis) to a binomial PMF (vertical axis) and includes a second simulated somatic mutation model LR distribution 822 and a second simulated germline mutation model LR distribution 824. The second simulated somatic mutation model LR distribution 822 corresponds to a highest likelihood somatic mutation model Mₛₒₘ for the second mutation based on the second context, and the second simulated germline mutation model LR distribution 824 corresponds to a highest likelihood germline mutation model M_{germ} for the second mutation based on the second context. For example, the second simulated somatic mutation model LR distribution 822 and the second simulated germline mutation model LR distribution 824 represent the terms Yₛₒₘ and Y_{germ}, respectively, for the second mutation. The second simulated log likelihood ratio graph 806 further includes a second threshold 826 for classifying the second mutation and a second observed log likelihood ratio 828 (e.g., the log likelihood ratio 526 determined for the second mutation).

The second simulated somatic mutation model LR distribution 822 and the second simulated germline mutation model LR distribution 824 of the second simulated log likelihood ratio graph 806 have much less overlap than the first simulated somatic mutation model LR distribution 810 and the first simulated germline mutation model LR distribution 812 of the first simulated log likelihood ratio graph 802. The smaller overlap between the second simulated somatic mutation model LR distribution 822 and the second simulated germline mutation model LR distribution 824 indicates that the highest likelihood somatic mutation model Mₛₒₘ and the highest likelihood germline mutation model M_{germ} for the second mutation are less likely to produce a similar log likelihood ratio value. This is also indicated in the second theoretical performance graph 808. The second theoretical performance graph 808 includes a second curve 830 and the desired sensitivity 820. Similar to the first curve 818, each point on the second curve 830 represents a different potential threshold value setting (e.g., the term t described with respect to FIG. 5) for classifying the second mutation.

In the present example, the second threshold 826 is also set based on the desired sensitivity 820, e.g., so that the sensitivity of classifying the second mutation is not less than the desired sensitivity 820. The second threshold 826 is depicted as a point on the second curve 830, indicating that the false positive rate is relatively low at this threshold value. This is also reflected in the second simulated log likelihood ratio graph 806, as a relatively small portion of the second simulated germline mutation model LR distribution 824 is less than the second threshold 826. As such, even while classifying a somatic mutation with high (e.g., greater than 95%) sensitivity, a germline mutation is not likely to be classified as a somatic mutation (e.g., the FPR is less than 50%).

In the example depicted in the second simulated log likelihood ratio graph 806, the second threshold 826 is 0.16, and the second observed log likelihood ratio 828 is 2.01. Using these example values, the second mutation may be classified as germline because the second observed log likelihood ratio 828 is greater than the second threshold 826.

Together, the illustrative examples 800 demonstrate how a difference between the germline and somatic models varies based on the context of the mutation, and models that are more different have more favorable ROC curves (e.g., a greater area under the curve). Moreover, calculating the threshold value 528 on a per-mutation basis allows the threshold value 528 to be adjusted based on the context.

It is to be appreciated that although the first simulated log likelihood ratio graph 802 and the second simulated log likelihood ratio graph 806 are depicted as line graphs, the first simulated log likelihood ratio graph 802 and the second simulated log likelihood ratio graph 806 may be visualized in other ways, such as bar graphs. Moreover, the likelihood distributions, the log likelihood ratios, and the thresholds may be determined without explicitly visualizing the values in graphic form.

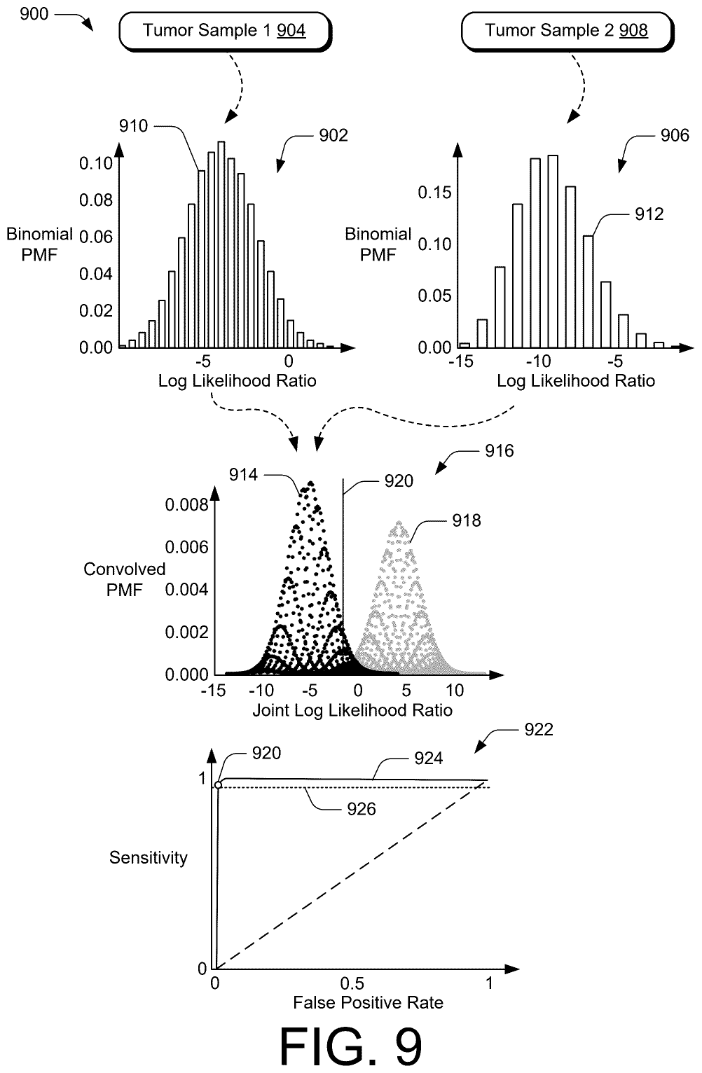

FIG. 9 depicts an illustrative example 900 of using a joint log likelihood ratio to determine a threshold for classifying a mutation observed in tumor sequencing data. The illustrative example 900 includes a first log likelihood ratio graph 902 derived from sequencing data obtained for a first tumor sample 904 (e.g., “Tumor Sample 1”) and a second log likelihood ratio graph 906 derived from sequencing data obtained for a second tumor sample 908 (e.g., “Tumor Sample 2”). The first tumor sample 904 and the second tumor sample 908 correspond to two samples obtained from a same individual, such as at different collection locations and/or collection times. The first log likelihood ratio graph 902 and the second log likelihood ratio graph 906 depict the log likelihood ratio (horizontal axis) with respect to binomial PMF (vertical axis) and are calculated for a same mutation using the sequencing data from the respective sample. The first log likelihood ratio graph 902 includes a first somatic mutation model log likelihood ratio distribution 910, which corresponds to the log likelihood ratio distribution (e.g., the term Yₛₒₘ described with respect to FIG. 5) of a somatic mutation model (e.g., the term Mₛₒₘ described with respect to FIG. 5) calculated for the first tumor sample 904. Similarly, the second log likelihood ratio graph 906 includes a second somatic mutation model log likelihood ratio distribution 912, which corresponds to the log likelihood ratio distribution of the same somatic mutation model as the first sample calculated for the second tumor sample 908. The first somatic mutation model log likelihood ratio distribution 910 and the second somatic mutation model log likelihood ratio distribution 912 are depicted as bar graphs, although other graph types are possible, such as line graphs.

The first somatic mutation model log likelihood ratio distribution 910 and the second somatic mutation model log likelihood ratio distribution 912 are summed to generate a joint somatic mutation model likelihood ratio distribution 914 of a joint log likelihood ratio graph 916. By way of example, the joint somatic mutation model likelihood ratio distribution 914 corresponds to the term JYₛₒₘ described above with respect to FIG. 5. The joint log likelihood ratio graph 916 further includes a joint germline mutation model likelihood ratio distribution 918 (e.g., the term JY_{germ} described above with respect to FIG. 5) and a joint threshold 920. For instance, although not explicitly shown in FIG. 9, a first germline mutation model log likelihood ratio distribution calculated for the first tumor sample 904 may be summed with a second germline mutation model log likelihood ratio distribution calculated for the second tumor sample 908.

The illustrative example 900 further depicts a theoretical performance graph 922, which relates a false positive rate (horizontal axis, corresponding to putative germline mutations that are classified as somatic) to sensitivity (vertical axis, corresponding to putative somatic mutations that are classified as somatic). The theoretical performance graph 922 includes a curve 924 and a desired sensitivity 926 (e.g., 95% sensitivity). The curve 924 is a ROC curve, such as described above with respect to the first curve 818 of FIG. 8. Each point on the curve 924 represents a different potential threshold value setting for classifying the mutation using the joint evidence from the first tumor sample 904 and the second tumor sample 908. The diagonal line from the bottom left to the top right represents random guessing.

In the present example, the joint threshold 920 is set based on the desired sensitivity 926. The joint threshold 920 is depicted as a point on the curve 924, indicating that the false positive rate is very low at this threshold value (e.g., close to 0%). By way of example, using the joint evidence and the joint threshold 920 rather than evaluating the log likelihood ratio calculated from the first tumor sample 904 sequencing data and the second tumor sample 908 sequencing data individually decreases the false positive rate while maintaining high sensitivity. By enabling data from multiple samples to be combined, the techniques described herein increase the statistical power for classifying mutations across the genome, even in tumor-only samples (e.g., samples with high purity) and samples with copy number alteration events that would otherwise be difficult to classify as germline or somatic.

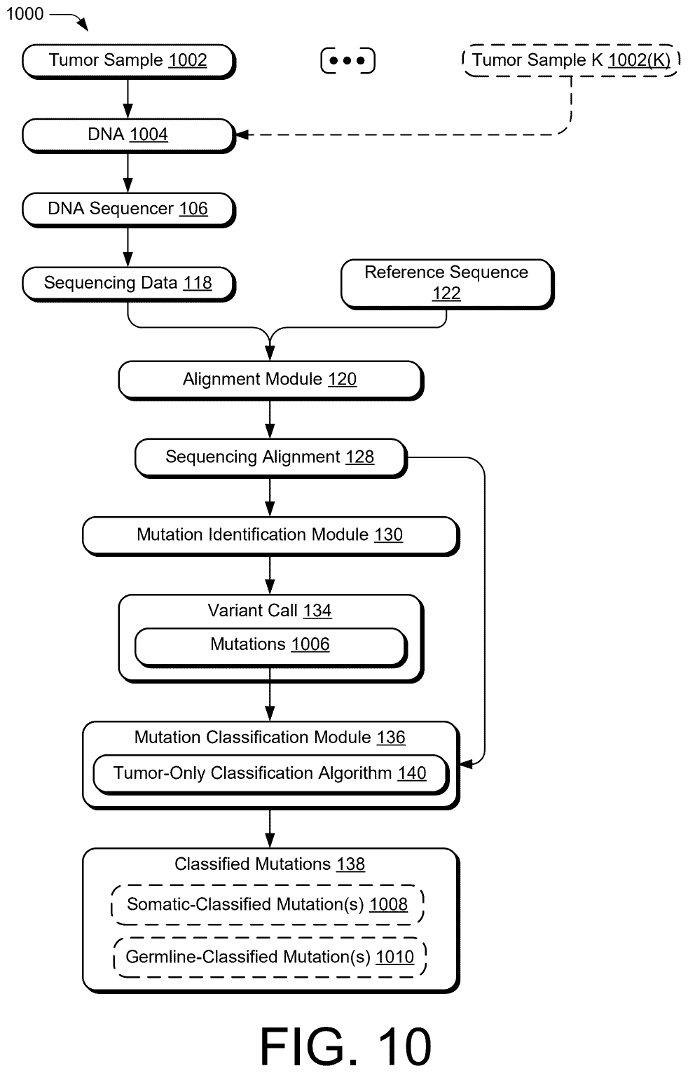

FIG. 10 depicts a workflow 1000 in an example implementation of using the mutation classification module 136 of FIG. 1 for classifying mutations as germline or somatic. For instance, the workflow 1000 outlines a mutation classification pipeline that will be described with reference to components previously introduced with respect to FIGS. 1, 2, and 5.

A tumor sample 1002 is processed to prepare a nucleic acid sample, shown in FIG. 10 as DNA 1004. By way of example, the DNA 1004 (or another nucleic acid) is isolated from the tumor sample 1002 using a DNA extraction technique. The tumor sample 1002 comprises, for example, blood, tissue, saliva, or another source of cells from an organism (e.g., an individual) of interest. In at least one variation, the tumor sample 1002 comprises cultured cells. Moreover, the DNA 1004 may be prepared for sequencing according to a protocol specified by a type of sequencing technique being used. This includes, for example, breaking the nucleic acid into fragments, amplifying the fragments, and/or adapting the fragments for sequencing.

Sequencing is performed by the DNA sequencer 106 to produce the sequencing data 118 from the DNA 1004. In at least one implementation, the DNA sequencer 106 employs fluorescence-based detection to determine an order of nucleotides in fragments of the DNA 1004. The sequencing data 118 comprises reads, which are ordered combinations of nucleotides, for the nucleic acid fragments.

As discussed above with respect to FIG. 1, the alignment module 120 receives the sequencing data 118 and maps the reads to the reference sequence 122 using the one or more alignment algorithms 126, resulting in the sequencing alignment 128. It is to be appreciated that as used herein, the term “align” and its conjugates is not limited to an exact 1:1 alignment between sequences. Rather, alignment is accomplished with a degree of accuracy that is adequate or desired based on its intended purpose (e.g., to map reads to the reference sequence 122 with a sufficient confidence and/or accuracy).

The mutation identification module 130 evaluates the sequencing alignment 128 to identify positions where a read does not match the reference sequence 122, e.g., using the one or more variant calling algorithms 132, and outputs the variant call 134. The variant call 134 includes mutations 1006, which correspond to genetic locations where a variant allele is detected by the mutation identification module 130.

The mutation classification module 136 receives data regarding the mutations 1006 from the sequencing alignment 128. For instance, the mutation classification module 136 may receive at least a portion of the sequencing alignment 128 that includes the mutations 1006 with annotations and/or information about their genomic location, the alternate count 208 versus the reference count 210, and so forth. In at least one implementation, the mutation classification module 136 utilizes the tumor-only classification algorithm 140 to classify the mutations 1006, such as when the tumor sample 1002 does not include a matched normal cell control sample. The tumor-only classification algorithm 140 may evaluate and classify individual mutations of the mutations 1006 separately.

As further described with respect to FIGS. 1 and 5, the copy profile interpretation algorithm 148 may determine the context 506 of the individual mutation of the mutations 1006, and the likelihood comparison algorithm 146 may determine whether the context 506 is better fit to one of the germline mutation models 142 or to one of the somatic mutation models 144. For example, the likelihood comparison algorithm 146 may compare the highest likelihood (e.g., best fitting) somatic mutation model to the highest likelihood (e.g., best fitting) germline mutation model using a ratio of the data likelihoods, e.g., the log likelihood ratio 526. The log likelihood ratio 526 may be compared to the threshold value 528, which may be zero or a non-zero value calculated per mutation. For instance, a similarity (or difference) between the highest likelihood somatic mutation model and the highest likelihood germline mutation model affects the threshold value 528. A particular mutation may be classified as somatic (e.g., not germline) in response to the log likelihood ratio 526 being less than the threshold value 528 or germline (e.g., not somatic) in response to the log likelihood ratio 526 being greater than or equal to the threshold value 528.

In at least one implementation, more than one tumor sample is obtained from the same individual, optionally indicated as a tumor sample K 1002 (K). There may be other tumor samples in addition to the tumor sample 1002 and the tumor sample K 1002 (K), as indicated by ellipses. The tumor sample K 1002 (K), as well as other tumor samples obtained from the individual, may be sequenced in a similar manner as the tumor sample 1002, and the mutation classification module 136 may receive the corresponding sequencing alignment(s) in order to calculate a joint threshold for the threshold value 528 that combines evidence from the multiple samples. By way of example, different samples may have different contexts, such as different purities, and the joint evidence may produce a greater separation between the germline mutation models 142 and the somatic mutation models 144 than when the multiple samples are analyzed individually.

The mutation classification module 136 individually processes the mutations 1006 via the tumor-only classification algorithm 140 and outputs the classified mutations 138, which include somatic-classified mutation(s) 1008 and/or germline-classified mutation(s) 1010. In at least one implementation, the workflow 1000 includes filtering which mutations are displayed (e.g., via the display device 150) to a user. By way of example, the workflow 1000 may include functionality to filter out the germline-classified mutation(s) 1010 so that a non-germline subset of the classified mutations 138 is shown, e.g., the somatic-classified mutation(s) 1008. The classified mutations 138 may be further evaluated in downstream analyses to estimate tumor burden, identify novel cancer drivers, identify mutational signatures, and/or the like.

Having discussed example details of the techniques for context-specific tumor-only mutation classification, consider now example procedures to illustrate additional aspects of the techniques.

### Example Procedure

This section describes an example procedure for context-specific tumor-only mutation classification in one or more implementations. Aspects of the procedure may be implemented in hardware, firmware, software, or a combination thereof. The procedure is shown as a set of blocks that specify operations performed by one or more devices and is not necessarily limited to the orders shown for performing the operations by the respective blocks. In at least some implementations, the procedure is performed by a suitably configured device, such as the sequencing data processor 108 of FIG. 1.

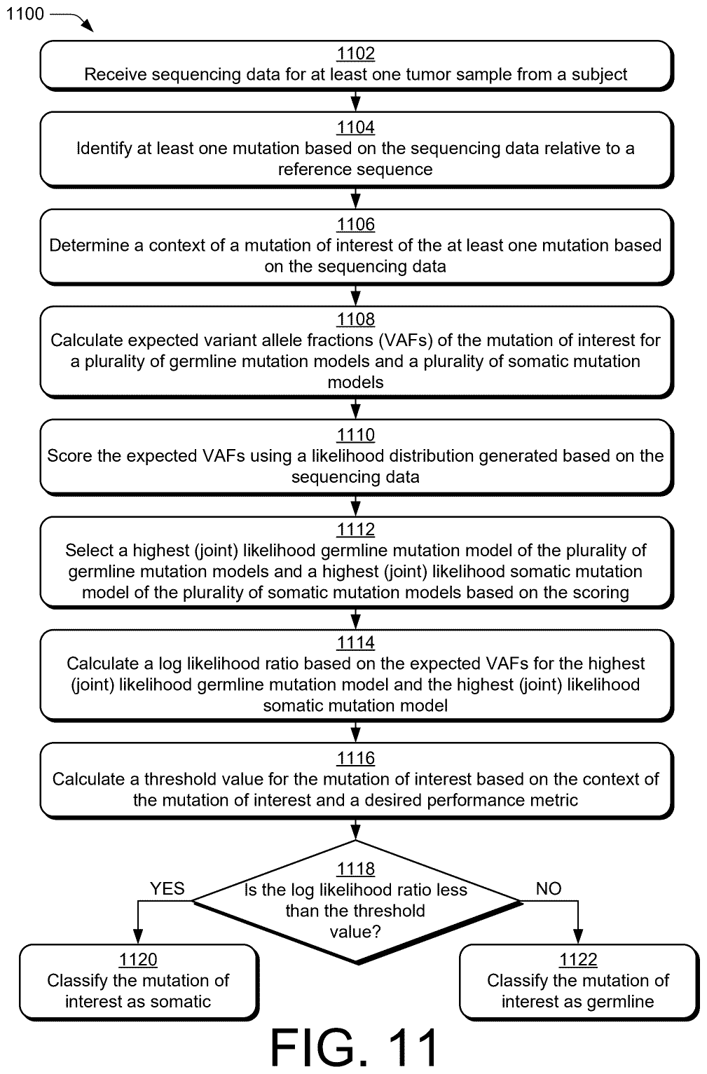

FIG. 11 depicts an example procedure 1100 in which context-specific tumor-only mutation classification is performed.

Sequencing data for at least one tumor sample from a subject are received (block 1102). By way of example, the at least one tumor sample comprises tumor (e.g., cancer) cells in a mixture with an unknown quantity of normal (e.g., non-cancerous) cells. The sequencing data (e.g., the sequencing data 118 of FIG. 1) are generated by a DNA sequencer (e.g., the DNA sequencer 106 of FIG. 1) on DNA prepared from respective tumor samples of the at least one tumor sample and may comprise short read sequencing data or long read sequencing data, depending on a specific DNA sequencing technique used. By way of example, the DNA sequencer may use a short read sequencing technique that produces sequence fragments typically ranging from approximately 10 bases to approximately 1000 bases and more typically from approximately 50 bases to approximately 500 bases. Alternatively, the DNA sequencer may use a long read sequencing technique that produces sequence fragments that typically range from 1000 bases to 1,000,000 bases and more typically from 5000 bases to 500,000 bases in length. In at least one implementation, the sequencing includes whole-exome sequencing, where protein-coding regions of the genome are sequenced.

At least one mutation is identified based on the sequencing data relative to a reference sequence (block 1104). By way of example, an alignment module (e.g., the alignment module 120 of FIG. 1) uses one or more alignment algorithms (e.g., the one or more alignment algorithms 126 of FIG. 1) to map the sequencing reads to locations in the genome using the reference sequence (e.g., the reference sequence 122 of FIG. 1). The one or more alignment algorithms include functionality for finding an alignment that increases (e.g., maximizes) a similarity between a read and the reference sequence using a scoring system that considers possible insertions, deletions, and mismatches. Aligning the sequencing reads to the reference genome generates a sequencing alignment (e.g., the sequencing alignment 128 of FIG. 1), which comprises sequence fragments (e.g., the sequencing reads) that have been successfully mapped to the reference sequence.

The at least one mutation may be identified at a particular region of the genome where multiple reads deviate from the reference sequence. For instance, the at least one mutation may be supported by an alternate count (e.g., the alternate count 208 of FIG. 2) of reads that contain an alteration in the sequence compared to the reference sequence, such as further described with respect to FIG. 2.

A context of a mutation of interest of the at least one mutation is determined based on the sequencing data (block 1106). By way of example, the sequencing data of a given tumor sample may be analyzed by a mutation classification module (e.g., the mutation classification module 136 of FIG. 1) on a per-mutation basis to determine the context, e.g., via a copy profile interpretation algorithm (e.g., the copy profile interpretation algorithm 148 of FIG. 1). The context describes properties of the given tumor sample as well as alterations at the local region of the mutation of interest. In at least one implementation, the context includes a purity of the given tumor sample (e.g., the purity 508 of FIG. 5), a ploidy of the given tumor sample's genome (e.g., the ploidy 510 of FIG. 5), a copy number alteration of the specific region of the mutation of interest (e.g., the CNA 512 of FIG. 5), a cancer cell fraction that includes the copy number alteration (e.g., the CCF of the CNA 514 of FIG. 5), and/or a cancer cell fraction that includes the mutation of interest (e.g., the CCF of the mutation 516 of FIG. 5).

In one or more implementations, the copy profile interpretation algorithm is configured to analyze read-depth information from the sequencing alignment and generate candidate copy profile interpretations of the corresponding tumor sample that enable the copy number alteration to be inferred in a genetic location-specific manner. This enables different copy number alterations to be inferred for different mutations of interest. The copy profile interpretation algorithm may be further configured to select respective values for the purity and the ploidy of the given tumor sample from the candidate solutions based in part on how well those values fit raw copy number data (e.g., the best-fitting values are selected).

For a given purity and ploidy solution, the copy profile interpretation algorithm may be further configured to infer the cancer cell fraction of the copy number alteration, which refers to a fraction of the cancer (e.g., tumor) cells in the corresponding tumor sample that contain the copy number alteration, and the cancer cell fraction of the mutation, which refers to the fraction of the cancer cells in the corresponding tumor sample that include the mutation of interest. These values represent the heterogeneity of the corresponding tumor sample, as different subclones may have different mutations, duplications, and/or deletions, for instance.

Expected variant allele fractions (VAFs) of the mutation of interest are calculated for a plurality of germline mutation models and a plurality of somatic mutation models (block 1108). By way of example, the plurality of germline mutation models (e.g., the germline mutation models 142 of FIG. 1) model different ways in which the mutation of interest can be observed (e.g., as alternate counts) within the corresponding sequencing data based on how the mutation is inherited (e.g., from one parent or both parents) and whether there are copy number variations. Similarly, the plurality of somatic mutation models (e.g., the somatic mutation models 144 of FIG. 1) model different ways in which the mutation of interest can be observed within the corresponding sequencing data based on when the mutation arises with respect to copy number alteration events.

The expected VAFs are calculated as a function of the context, such as described with respect to FIGS. 6A-6C. Accordingly, separate VAFs may be calculated for respective tumor samples of the at least one tumor sample for the plurality of germline mutation models and the plurality of somatic mutation models. Moreover, the VAFs are specific to the mutation of interest.

The expected VAFs are scored using a likelihood distribution generated based on the sequencing data (block 1110). By way of example, the mutation classification module may utilize a likelihood comparison algorithm (e.g., the likelihood comparison algorithm 146 of FIG. 1) to generate the likelihood distribution (e.g., the likelihood distribution 524 of FIG. 5) based on the corresponding sequencing data of the mutation of interest. In at least one implementation, the likelihood comparison algorithm uses a beta binomial distribution to model the distribution of the corresponding sequencing data. For instance, because there is a discrete number of cells and units of the genome in the corresponding tumor sample, the beta binomial distribution is a statistical model that describes the distribution of the true measurement (e.g., the true number of alternate counts, which may differ from the observed number of alternate counts) of the mutation of interest.

In at least one implementation, the likelihood comparison algorithm may score a given expected VAF by calculating a likelihood (e.g., a conditional probability) of observing the given expected VAF according to the likelihood distribution. The likelihood corresponds to the probability of the corresponding model explaining the sequencing data, for instance.

When multiple tumor samples are present, the likelihood comparison algorithm may generate separate likelihood distributions for respective tumor samples of the at least one tumor sample and separately score the corresponding expected VAFs accordingly. Then, a joint likelihood value is calculated for each mutation model (germline and somatic) across all the tumor samples.

A highest (joint) likelihood germline mutation model of the plurality of germline mutation models and a highest (joint) likelihood somatic mutation model of the plurality of somatic mutation models are selected based on the scoring (block 1112). By way of example, the likelihood comparison algorithm may identify the highest (joint) likelihood germline mutation model as the model having the greatest (joint) likelihood of the plurality of germline mutation models for the mutation of interest. Similarly, the likelihood comparison algorithm may identify the highest (joint) likelihood somatic mutation model as the model having the greatest (joint) likelihood of the plurality of somatic mutation models for the mutation of interest.

A log likelihood ratio is calculated based on the expected VAFs for the highest (joint) likelihood germline mutation model and the highest (joint) likelihood somatic mutation model (block 1114). By way of example, the likelihood comparison algorithm may compute the log likelihood ratio (e.g., the log likelihood ratio 526 of FIG. 5) as the logarithm of the likelihood of the highest likelihood somatic mutation model (e.g., as determined based on the expected VAF of the highest likelihood somatic mutation model and the corresponding sequencing data) subtracted from the logarithm of the likelihood of the highest likelihood germline mutation model (e.g., as determined based on the expected VAF(s) of the highest (joint) likelihood germline mutation model and the corresponding sequencing data). Alternatively, the likelihood comparison algorithm may compute the log likelihood ratio as a logarithm of the likelihood of the highest (joint) likelihood germline mutation model divided by the likelihood of the highest (joint) likelihood somatic mutation model.

As indicated above, when multiple tumor samples are present, the likelihood comparison algorithm may sum the log likelihood distributions of the highest likelihood somatic mutation model for respective tumor samples and sum the log likelihood distributions of the highest likelihood germline mutation model for the respective tumor samples, resulting in joint log likelihood distributions, such as depicted in the joint log likelihood ratio graph 916 of FIG. 9. The log likelihood ratio may then be determined from the joint log likelihood distributions, resulting in a joint log likelihood ratio that combines evidence from the multiple tumor samples.

A threshold value is calculated for the mutation of interest based on the context of the mutation of interest and a desired performance metric (block 1116). In general, a negative value for the log likelihood ratio indicates that the highest likelihood somatic mutation model better fits the corresponding sequencing data, whereas a positive value for the log likelihood ratio indicates that the highest likelihood germline mutation model better fits the data. Thus, in some instances, the threshold value (e.g., the threshold value 528 of FIG. 5) is set to zero. However, in other instances, zero may lead to low sensitivity for true somatic mutations, such as when the highest likelihood germline mutation model and the highest likelihood somatic mutation model have similar or near-equal expected VAFs. Therefore, in at least one implementation, the likelihood comparison algorithm calculates the threshold value as a function of the context of the mutation of interest and the desired classification performance metric.

As mentioned above, the highest likelihood somatic mutation model and the highest likelihood germline mutation models have corresponding expected VAFs. The expected alternate count supporting the mutation of interest follows a binomial distribution that is a function of the expected VAF of the corresponding model and a sequencing depth (e.g., a total number of reads at the particular region of the genome containing the mutation of interest). For each possible expected alternate count value, the likelihood comparison algorithm may further calculate an expected log likelihood to generate a distribution representing the log likelihood ratio that the mutation of interest was truly sampled from the corresponding model based on the context. This log likelihood ratio distribution may be separately computed for the highest likelihood somatic mutation model and the highest likelihood germline mutation model, such as elaborated above with respect to FIGS. 5 and 9.

The threshold value may be calculated based on the desired performance metric using the log likelihood ratio distributions of the highest likelihood somatic mutation model and/or the highest likelihood germline mutation model. For example, the desired performance metric may be a targeted or acceptable sensitivity of classifying somatic mutations as somatic (e.g., a sensitivity value in a range from 90-99%, such as 95%), and the threshold value may be calculated using the log likelihood ratio distribution of the highest likelihood somatic mutation model. Additionally, the corresponding expected false positive rate may be calculated and reported for the mutation. In such a scenario, the threshold value may correspond to a value that minimizes a difference between the targeted or acceptable sensitivity and a cumulative sum of the log likelihood ratio distribution of the highest likelihood somatic mutation model.

Alternatively, the desired performance metric may be a targeted or acceptable false positive rate of classifying germline mutations as somatic (e.g., a false positive rate in a range from 0-50%), and the threshold value may be calculated using the log likelihood ratio distribution of the highest likelihood germline mutation model. Additionally, the corresponding sensitivity may be calculated and reported for the mutation. In such a scenario, the threshold value may correspond to a value that minimizes a difference between the targeted or acceptable false positive rate and a cumulative sum of the log likelihood ratio distribution of the highest likelihood germline mutation model.

When multiple tumor samples are present, the likelihood comparison algorithm may use the joint log likelihood distributions of the respective models, resulting in a joint threshold value that combines evidence from the multiple tumor samples.

It is determined if the log likelihood ratio is less than the threshold value (block 1118). By way of example, the likelihood comparison algorithm may compare the log likelihood ratio (or the joint log likelihood ratio when multiple tumor samples are present) to the threshold value to determine the mutation classification of the mutation of interest.

If the log likelihood ratio is less than the threshold value, the mutation of interest is classified as somatic (block 1120). By way of example, because the log likelihood ratio is less than the threshold value, the highest likelihood somatic mutation model is a better fit for the corresponding sequencing data than the highest likelihood germline mutation model. In at least one implementation, the mutation of interest is putatively labeled as a somatic mutation within a list of classified mutations (e.g., the classified mutations 138 of FIG. 1), which may be output for downstream analyses.

If the log likelihood ratio is not less than the threshold value, the mutation of interest is classified as germline (block 1122). By way of example, because the log likelihood ratio is greater than or equal to the threshold value, the highest likelihood germline mutation model is a better fit for the corresponding sequencing data than the highest likelihood somatic mutation model. In at least one implementation, the mutation of interest is putatively labeled as a germline mutation within the list of classified mutations.

In this way, the procedure 1100 uses the context of a mutation to determine whether it is more likely to be germline or somatic according to a context-based threshold that is set based on a desired performance metric (e.g., a true positive rate/sensitivity or a false positive rate). As a result, the at least one mutation is more accurately classified and includes interpretable expected classification performance, which aids in downstream analysis of biological mechanisms that drive cancer development and progression, for example.

Having described an example procedure in accordance with one or more implementations, consider now an example system and device that can be utilized to implement the various techniques described herein.

### Example System and Device

FIG. 12 illustrates an example system generally at 1200 that includes an example computing device 1202 that is representative of one or more computing systems and/or devices that may implement the various techniques described herein. This is illustrated through inclusion of the sequencing data processor 108. The computing device 1202 may be, for example, a server of a service provider, a device associated with a client (e.g., a client device), an on-chip system, and/or any other suitable computing device or computing system.

The example computing device 1202 as illustrated includes a processing system 1204, one or more computer-readable media 1206, and one or more I/O interfaces 1208 that are communicatively coupled, one to another. Although not shown, the computing device 1202 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1204 is representative of functionality to perform one or more operations using hardware. Accordingly, the processing system 1204 is illustrated as including hardware elements 1210 that may be configured as processors, functional blocks, and so forth. This may include implementation in hardware as an application specific integrated circuit or other logic device formed using one or more semiconductors. The hardware elements 1210 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable media 1206 is illustrated as including memory/storage 1212. The memory/storage 1212 represents memory/storage capacity associated with one or more computer-readable media. The memory/storage 1212 may include volatile media (such as random-access memory (RAM)) and/or nonvolatile media (such as read only memory (ROM), Flash memory, optical disks, magnetic disks, and so forth). The memory/storage 1212 may include fixed media (e.g., RAM, ROM, a fixed hard drive, and so on) as well as removable media (e.g., flash memory, a removable hard drive, an optical disc, and so forth). The computer-readable media 1206 may be configured in a variety of other ways as further described below.

Input/output interface(s) 1208 are representative of functionality to allow a user to enter commands and information to computing device 1202 and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1202 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hard-wired device that performs operations based on hard-wired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 1202. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1202, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1210 and computer-readable media 1206 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1210. The computing device 1202 may be configured to implement particular instructions and/or functions corresponding to the software and/or hardware modules. Accordingly, implementation of a module that is executable by the computing device 1202 as software may be achieved at least partially in hardware, e.g., through use of computer-readable storage media and/or hardware elements 1210 of the processing system 1204. The instructions and/or functions may be executable/operable by one or more articles of manufacture (for example, one or more computing devices 1202 and/or processing systems 1204) to implement techniques, modules, and examples described herein.

The techniques described herein may be supported by various configurations of the computing device 1202 and are not limited to the specific examples of the techniques described herein. This functionality may also be implemented all or in part through use of a distributed system, such as over a “cloud” 1214 via a platform 1216 as described below.

The cloud 1214 includes and/or is representative of a platform 1216 for resources 1218, which are depicted including the sequencing data processor 108. The platform 1216 abstracts underlying functionality of hardware (e.g., servers) and software resources of the cloud 1214. The resources 1218 may include applications and/or data that can be utilized while computer processing is executed on servers that are remote from the computing device 1202. Resources 1218 can also include services provided over the Internet and/or through a subscriber network, such as a cellular or Wi-Fi network.

The platform 1216 may abstract resources and functions to connect the computing device 1202 with other computing devices. The platform 1216 may also serve to abstract scaling of resources to provide a corresponding level of scale to encountered demand for the resources 1218 that are implemented via the platform 1216. Accordingly, in an interconnected device embodiment, implementation of functionality described herein may be distributed throughout the system 1200. For example, the functionality may be implemented in part on the computing device 1202 as well as via the platform 1216 that abstracts the functionality of the cloud 1214.

### CONCLUSION

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. A system for context-specific mutation classification, comprising:
a mutation classification module implemented in a non-transitory computer-readable storage medium and configured to:
classify a mutation identified in sequencing data from a tumor sample as germline or somatic based on a likelihood ratio relative to a threshold that is calculated based on a context of the mutation, the likelihood ratio comparing a germline model likelihood of a germline model of the mutation to a somatic model likelihood of a somatic model of the mutation; and
output the classification of the mutation.

2. The system of claim 1, wherein the germline model is selected from a plurality of germline models based on a fit of the germline model to the sequencing data relative to other germline models of the plurality of germline models, and the somatic model is selected from a plurality of somatic models based on a fit of the somatic model to the sequencing data relative to other somatic models of the plurality of somatic models.

3. The system of claim 2, wherein the mutation classification module is further configured to:
generate a likelihood distribution of a true measurement of alternate counts for the mutation based on the sequencing data;
determine expected variant allele fractions of the mutation for the plurality of germline models and the plurality of somatic models based on the context of the mutation;
determine respective fits of the plurality of germline models and the plurality of somatic models to the likelihood distribution based on the expected variant allele fractions;
select the germline model based on the respective fits of the plurality of germline models; and
select the somatic model based on the respective fits of the plurality of somatic models.

4. The system of claim 3, wherein the likelihood distribution is generated using a beta binomial distribution.

5. The system of claim 1, wherein the mutation classification module is further configured to:
determine the context of the mutation based on copy number data of the sequencing data, the context comprising a purity of the tumor sample and a ploidy of the tumor sample.

6. The system of claim 5, wherein, to determine the context of the mutation based on the copy number data of the sequencing data, the mutation classification module is configured to:
generate candidate copy profile interpretations comprising different values for the purity of the tumor sample and the ploidy of the tumor sample based on the copy number data; and
select a copy profile interpretation of the candidate copy profile interpretations based on a fit of the copy profile interpretation to the copy number data, wherein the purity of the tumor sample corresponds to a purity value of the selected copy profile interpretation and the ploidy of the tumor sample corresponds to a ploidy value of the selected copy profile interpretation.

7. The system of claim 5, wherein the context further comprises a copy number alteration at a genetic location of the mutation, a first cancer cell fraction that includes the copy number alteration, and a second cancer cell fraction that includes the mutation, and wherein the mutation classification module is further configured to:
infer each of the copy number alteration, the first cancer cell fraction, and the second cancer cell fraction based on the purity of the tumor sample, the ploidy of the tumor sample, and the copy number data.

8. The system of claim 1, wherein to classify the mutation, the mutation classification module is configured to:
classify the mutation as somatic in response to a logarithm of the likelihood ratio being less than the threshold; or
classify the mutation as germline in response to the logarithm of the likelihood ratio being greater than or equal to the threshold.

9. The system of claim 1, wherein the germline model likelihood is a sum of germline model likelihood distributions determined for a plurality of tumor samples from a same subject, the plurality of tumor samples including the tumor sample, and the somatic model likelihood is a sum of somatic model likelihood distributions determined for the mutation from the plurality of tumor samples.

10. The system of claim 1, wherein the mutation classification module is further configured to:
calculate the threshold based on the context of the mutation and further based on a desired performance metric and the somatic model of the mutation or the germline model of the mutation.

11. The system of claim 10, wherein:
the threshold is calculated based on the somatic model of the mutation in response to the desired performance metric being a target sensitivity for classifying somatic mutations as somatic; or
the threshold is calculated based on the germline model of the mutation in response to the desired performance metric being a target false positive rate for classifying germline mutations as somatic.

12. A method for context-specific mutation classification, comprising:
receiving a sequencing alignment for a tumor sample, the sequencing alignment comprising a plurality of sequencing reads aligned to a reference sequence;
identifying a mutation at a genetic region where at least a subset of the plurality of sequencing reads differs from the reference sequence;
classifying the mutation as germline or somatic based on a log likelihood ratio relative to a threshold, the log likelihood ratio indicating a relative fit of a germline mutation model of the mutation and a somatic mutation model of the mutation to data from the sequencing alignment based on a context of the mutation; and
outputting the classification of the mutation.

13. The method of claim 12, further comprising:
calculating the threshold based on the context of the mutation and further based on one of a desired sensitivity for classifying somatic mutations as somatic or a desired false positive rate for classifying germline mutations as somatic.

14. The method of claim 12, wherein the context comprises a purity of the tumor sample, a ploidy of the tumor sample, a copy number variation at the genetic region, a first fraction of cancer cells in the tumor sample that includes the copy number variation, and a second fraction of cancer cells in the tumor sample that includes the mutation.

15. The method of claim 12, further comprising:
selecting the germline mutation model from a set of germline mutation models based on a germline model likelihood of the germline mutation model relative to other germline mutation models of the set of germline mutation models; and
selecting the somatic mutation model from a set of somatic mutation models based on a somatic model likelihood of the somatic mutation model relative to other somatic mutation models of the set of somatic mutation models.

16. The method of claim 15, wherein selecting the germline mutation model from the set of germline mutation models further comprises:
computing germline model likelihoods for respective germline mutation models based on respective expected variant allele fractions for the respective germline mutation models and the data from the sequencing alignment; and
selecting the germline mutation model having a greatest germline model likelihood of the germline model likelihoods.

17. The method of claim 15, wherein selecting the somatic mutation model from the set of somatic mutation models further comprises:
computing somatic model likelihoods for respective somatic mutation models based on respective expected variant allele fractions of the respective somatic mutation models and the data from the sequencing alignment; and
selecting the somatic mutation model having a greatest somatic model likelihood of the somatic model likelihoods.

18. A method for context-specific mutation classification, comprising:
receiving sequencing alignments for a plurality of tumor samples obtained from an individual, the sequencing alignments comprising a plurality of sequencing reads aligned to a reference sequence for individual tumor samples of the plurality of tumor samples;
identifying a mutation at a genetic region where at least a subset of the plurality of sequencing reads differs from the reference sequence for the plurality of tumor samples;
separately calculating log likelihood ratio distributions for individual samples of the plurality of tumor samples, each log likelihood ratio distribution indicating a relative fit of a germline model of the mutation and a somatic model of the mutation to data from a corresponding sequencing alignment;
classifying the mutation as germline or somatic based on a joint log likelihood ratio for the plurality of tumor samples relative to a joint threshold, the joint log likelihood ratio being a sum of the log likelihood ratio distributions of the individual samples; and
outputting the classification of the mutation.

19. The method of claim 18, further comprising calculating the joint threshold based on a context of the mutation and a desired performance metric for classifying the mutation, and wherein classifying the mutation as germline or somatic based on the joint log likelihood ratio relative to the joint threshold comprises:
classifying the mutation as germline in response to the joint log likelihood ratio being greater than or equal to the joint threshold; or
classifying the mutation as somatic in response to the joint log likelihood ratio being less than the joint threshold.

20. The method of claim 19, wherein:
calculating the joint threshold is further based on a sum of germline log likelihood ratio distributions for the plurality of tumor samples in response to the desired performance metric being a desired false positive rate for classifying germline mutations as somatic; or
calculating the joint threshold is further based on a sum of somatic log likelihood ratio distributions for the plurality of tumor samples in response to the desired performance metric being a desired true positive rate for classifying somatic mutations as somatic.
